(19) United States
(12) Patent Application Publication
(10) Pub. No.: US 2010/0047334 A1
(43) Pub. Date: Feb. 25, 2010

(54) PH SENSITIVE LIPOSOME COMPOSITIONS FOR CONTROLLING SURFACE TOPOGRAPHY AND BINDING REACTIVITY IN FUNCTIONALIZED LIPOSOMES

(76) Inventor: Sofou Stavroula, New York, NY (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/511,110

(22) Filed: Jul. 29, 2009

Related U.S. Application Data
(63) Continuation-in-part of application No. 12/443,496.
(60) Provisional application No. 60/828,523, filed on Oct. 6, 2006.

(51) Int. Cl. A61K 9/127; A61K 47/44

(52) U.S. Cl. 424/450; 514/784

(57) ABSTRACT

Methods for controlling surface topography and binding reactivity in functionalized lipid layers, including in the form of liposomes, using pH-dependent processes. During direct cell-to-cell communication, lipids on the extracellular side of plasma membranes reorganize, and membrane associated communication-related molecules co-localize. At co-localization sites, sometimes identified as rafts, the local cell surface topography and reactivity are altered. Integration of these processes on nanometer-sized lipid vesicles used as drug delivery carriers would precisely control their interactions with diseased cells minimizing toxicities. Included are pH-dependent processes on functionalized lipid bilayers demonstrating reversible sharp changes in binding reactivity within a narrow pH window. Cholesterol enables tuning of the membrane reorganization to occur at pH values not necessarily close to the reported pKa's of the constituent titratable lipids. One illustrative function of the invention is to use liposomes to deliver bioactive agents to cancer or tumor cells and compositions of specific lipids that form liposomes to deliver a biologically active agent.

### STATEMENT OF RELATED APPLICATIONS

This application is based on and claims the benefit of U.S. patent application Ser. No. 12/443,496 having a filing date of 30 Mar. 2009, currently pending, which is based on and claims the benefit of Patent Cooperation Treaty (PCT) International Application No. PCT/US2007/080614 having an International Filing Date of 5 Oct. 2007, which is based on and claims the benefit of U.S. Provisional Patent Application No. 60/828,523 having a filing date of 6 Oct. 2006.

### BACKGROUND OF THE INVENTION

1. Technical Field

This invention relates to the field of pH-dependent formation of liquid heterogeneities and controlling the surface topography and binding reactivity in functionalized lipid bilayers. This invention also relates to the field of therapeutic delivery systems and liposome compositions. Further, this invention relates to the field of compositions of lipids that form liposomes, which can deliver a biologically active agent.

2. Related Art

The structural component of a cell membrane is a lipid bilayer. There is increasing evidence that critical cell functions are strongly correlated with reorganization of membranes into lipid rafts although the question whether membrane rafts are functionally relevant is still a controversial one. Lipid rafts are defined as nanometer- to micron-size lipid domains of laterally phase separated lipids. They are suggested to be involved in biological events including membrane trafficking, cell signaling, and viral infection mechanisms. During these events, co-localization of membrane proteins and of other macromolecules occurs on the surface of cells. During direct cell-to-cell communication, lipids on the extracellular side of plasma membranes reorganize, and membrane associated communication-related molecules co-localize. At co-localization sites, sometimes identified as rafts, the local cell surface topography and reactivity are altered. The processes regulating these changes are largely unknown.

Lipidic particles can be complexed with virtually any biological material. This capability allows these lipidic particles to be used as delivery systems for bioactive agents. Lipidic complexes have been used for a myriad of drug therapies, and one area in which these delivery systems have shown promising results is in cancer therapies. For a cancer therapy to be successful and efficient, the bioactive agent should be targeted to the tumor or cancer cell.

In some cases, only after the drug carriers are localized within the tumor interstitium, cancer-targeting ligands are necessary to enhance binding of the carriers to cancer cells, and to mediate their cellular internalization that increases drug bioavailability. At other times, during circulation in the bloodstream, ‘decoration’ of the carrier surface with tumor-binding ligands can activate non-desirable interactions with the host's immune- and reticuloendothelial- (RES) systems resulting in fast removal of the carriers from the blood stream, in low tumor absorbed doses, and in accumulation of drug carriers in healthy organs where release of therapeutic contents will kill healthy cells and increase toxicity.

Accordingly, there is always a need for an improved liposome for delivering bioactive agents. Additionally, there is always a need for an improved liposome that can be targeted to tumors and cancer cells. Further there is a need for an improved liposome that can be minimally recognized by the reticuloendothelial and immune systems. It is to these needs, among others, that this invention is directed.

### BRIEF SUMMARY OF THE INVENTION

On model lipid membranes, simplified processes that control surface topography and reactivity may potentially contribute to the understanding and control of related cell functions and associated diseases. Integration of these processes on nanometer-sized lipid vesicles used as drug delivery carriers would precisely control their interactions with diseased cells minimizing toxicities. Briefly, the present invention comprises such basic pH-dependent processes on model functionalized lipid bilayers, demonstrating reversible sharp changes in binding reactivity within a narrow pH window. Cholesterol enables tuning of the membrane reorganization to occur at pH values not necessarily close to the reported pka's of the constituent titratable lipids, and bilayer reorganization over repeated cycles of induced pH changes exhibits hysteresis.

The structural component of a cell membrane is a lipid bilayer. There is increasing evidence that critical cell functions are strongly correlated with reorganization of membranes into lipid rafts although the question whether membrane rafts are functionally relevant is still a controversial one. Lipid rafts are defined as nanometer- to micron-size lipid domains of laterally phase separated lipids. Lipid rafts are suggested to be involved in biological events including membrane trafficking, cell signaling, and viral infection mechanisms. During these events, co-localization of membrane proteins and of other macromolecules occurs on the surface of cells, therefore changing the effective reactivity and possibly resulting in remodeling of the cell surface topography.

In the present invention, the lipid bilayer membrane is used as the structural foundation whose lateral reorganization into lipid heterogeneities affects the lateral localization of reactive molecules that are attached to it, with implications in the effective reactivity of the membrane. The present invention uses an external stimulus such as pH to reorganize a model, functionalized lipid bilayer membrane into lipid heterogeneities. Information from the current understanding of molecular interactions that drive lipid phase separation in model membranes is used as background.

In particular examples, using pH as a trigger, lipid heterogeneities occurring in complex bilayer membranes are formed in the form of vesicles containing (i) a “domain” forming lipid with titratable anionic headgroups, (ii) a lipid A with grafted polymer chains (e.g., DSPE-PEG), (iii) non-ionizable lipid B with hydrocarbon tails that are different from or the same length as those in lipid A (e.g., DPPC), and (iv) a lipid with grafted functional groups and hydrocarbon tails identical to lipid B (e.g., DPPE-biotin). Lowering the pH creates lipid phase separation on the membrane. At high pH values, the lipid A headgroups are charged and repulsion between the headgroups makes the lipid energetically less likely to crystallize. The membrane appears spatially less heterogeneous, and the functional groups are obstructed by surrounding polymer chains. As the pH value is lowered, the anionic A headgroups become protonated, reducing electrostatic repulsion while possibly increasing hydrogen bonding between newly protonated A headgroups. These conditions favor phase separation in which the polymer-conjugated lipids potentially partition into the newly protonated lipid heterogeneities, driven by the dispersive attractive forces between hydrocarbon tails of the same length or fluidity state. In contrast, the functionalized lipids of the type B preferentially partition in the areas of lipids of the type B that are depleted in polymer lipids. Therefore, functionalized lipids become exposed and available to interact with their targets (white squares containing binding pockets in FIG. 1) increasing the effective binding reactivity of membranes.

This process of the present invention causes reversible reorganization of the bilayer into lipid heterogeneities possibly resulting in remodeling of its surface topography. This process also alters reversibly the membrane's binding reactivity towards its molecular targets. For example, membrane functionality is introduced by biotinylated lipids. The membrane's pH-dependent binding reactivity is evaluated towards streptavidin-covered microparticles, as an example for target, within the pH range of 7.4 and 6.7. These pH values correspond to the physiological pH of blood and the interstitial pH of cancerous tumors, respectively. Solid tumors often exhibit a pH gradient between the physiologic pH at perivascular regions and their more acidic core.

The 7.4 to 6.7 pH range was chosen as an example based on the rationale that these pH-dependent heterogeneous membranes in the form of vesicles may be utilized as targeted drug delivery carriers to advanced vascularized tumors to minimize toxicities and maximize tumor penetration using the following two mechanisms. First, since the molecular targets used for targeted cancer therapy are usually not unique to cancer cells, hiding of the targeting ligands from the vesicle surface during their circulation in the blood (at pH 7.4) may decrease toxicities arising from binding to healthy sites, while exposure of targeting ligands after extravasation in the tumor interstitial space (at pH 6.7) may increase vesicle binding to and uptake by cancer cells. Second, slow diffusion of vesicles in the tumor interstitial space combined with fast internalization rates of antibody-labeled vesicles by tumor cells, fast recycling of targeted antigens, and, fast systemic clearance of vesicles from circulation decreases the penetration depth of these carriers into the tumors. These targeted vesicles with pH-dependent binding reactivity should exhibit increasingly higher reactivity with cancer cells as they diffuse deeper into the more acidic tumor interstitium resulting in greater penetration within the tumor.

One illustrative application of the invention relates to liposomes that are able to be tuned to ‘hide’ (or ‘mask’) the targeting ligands during circulation and to ‘expose’ the targeting ligands after the liposomes extravasate into the (acidic) tumor interstitium where the liposomes are in the close vicinity of cancer cells. These liposomes can effectively address the issue of toxicity of immunoreactivity. This invention includes types of liposomes that can circulate for longer periods of time in the blood stream and can be absorbed by tumors after accumulation within the tumor interstitium, to result in internalization by solid-tumor cancer cells with less identification by the immune- and RES-systems. These liposomes can exhibit high tumor accumulation and high drug bioavailability in vivo within tumor cells. In one embodiment, the liposomes can comprise ionizable ‘domain-forming’ (‘raft’-forming) rigid lipids that are triggered to form lipid-phase separated domains in response to the tumor interstitial acidic pH (e.g. 6.7) environment. The liposomal membrane can be composed of rigid lipids and PEGylated lipids so as to increase the blood circulation times. Further, PEGylation may not interfere with the pH-sensitive properties of the developed liposomes, as the domain-forming property of rigid-lipids (each being lamellar-forming) can be utilized.

Tumor-targeting ligands can be conjugated on the headgroup of ‘raft’-forming lipids that preferentially partition into one type of domain after lipid-phase separation occurs at pH=6.7. Liposomes also contain PEGylated lipids that do not preferentially partition into the above mentioned domains after their formation. For example, at physiological pH of about 7.4 (e.g. in blood circulation) the lipids can be charged, the lipids composing the liposome membrane are ‘mixed’ on the plane of the membrane and are largely homogeneous, and the PEGylated lipids are uniformly distributed throughout the liposome membrane, thus adequately ‘masking’ (e.g. sterically hindering) the surface conjugated tumor-targeting ligands. As the pH is lowered, separated lipid domains are formed, in some of which tumor-targeting ligands are clustered and from which PEGylated lipids are excluded. As a result, the surface-conjugated ligands can be exposed with selectivity.

Using liposomes with targeting ligands that become ‘hidden’ or ‘exposed’ depending on the pH of their immediate environment, the fraction of liposomes that is internalized by cancer cells in vivo, after liposome extravasation into the tumor interstitium, can be dramatically increased within the cancer cells that constitute the metastatic vascularized tumors. This can allow for lower administered doses, and higher tumor adsorbed doses, which can result in lower toxicities. This illustrative application can be extrapolated to other types of cells.

These features, and other features and advantages of the present invention, will become more apparent to those of ordinary skill in the relevant art when the following detailed description of the preferred embodiments is read in conjunction with the appended drawings.

### BRIEF DESCRIPTION OF THE DRAWINGS

FIG. 1 is a diagrammatic illustration of the tunable surface topography and reactivity of lipid bilayers using pH as a trigger.

FIG. 2 is a series of graphs illustrating how cholesterol and the PEGylated lipid's acyl-tail length affect the extent of formation of membrane heterogeneities with pH. (2a) bilayers containing 5% mol cholesterol; (2b) bilayers not containing cholesterol; (2c) bilayers containing 5% mol cholesterol and 0.5% mol DSPE-PEG lipids; (2d) bilayers containing 5% mol cholesterol and 0.5% mol DPPE-PEG lipids.

FIG. 3 is a series of graphs illustrating how DSPE-PEG lipids content affects the extent of formation of membrane heterogeneities and their pH dependence. DSC scans of vesicles containing equimolar ratios of DPPC lipid and DSPS lipid with 5% mol cholesterol and variable fractions of DSPE-PEG lipid at pH values ranging from 7.4 to 6.5. (3a) 0.1% mol DSPE-PEG; (3b) 0.5% mol DSPE-PEG; (3c) 1.0% mol DSPE-PEG; (d) 1.5% mol DSPE-PEG.

FIG. 4 is a set of graphs illustrating the reversibility of the M/E ratio upon repeated introduction and removal of transmembrane pH gradients across the membranes of unilamellar vesicles. (4a) is after 2 hours; (4b) is after 4 hours.

FIG. 5 is a graph illustrating how pH-dependent vesicle binding reactivity is correlated with pH-dependent formation of lipid heterogeneities.

FIG. 6 is a Cryo-TEM image showing vesicle lamellarity.

FIG. 7 is a set of graphs illustrating how cholesterol affects the extent of formation of membrane heterogeneities with pH. (7a) shows bilayers not containing cholesterol; (7b) shows bilayers containing 5% mol cholesterol.

FIG. 8 is a series of graphs illustrating how pH-dependent changes in membrane's surface topography are demonstrated by pH-dependent vesicle binding reactivity. (8a) is 0.10% mol; (8b) is 0.25% mol; (8c) is 0.50% mol; (8d) is 0.75% mol; (8e) is 1.00% mol; (8f) is 1.50% mol.

FIG. 9 is a graph illustrating encapsulated content-to-lipid ratios in bound biotinylated vesicles.

FIG. 10 is a graph illustrating the absence of membrane permeability to calcein within the pH range of 7.4 and 5.5.

FIG. 11 is a graph Illustrating that the pH-controlled binding reactivity of biotinylated vesicles is retained in the presence of serum proteins.

FIG. 12 is a graph illustrating that the pH-dependent changes of the phenomenological lipid mobilities occur mostly within the first 24 hours upon introduction of pH gradients.

FIG. 13 is a graph illustrating that vesicles composed of pH-independent membranes do not exhibit pH-dependent binding reactivity wherein an increase in PEGylation lowers the binding reactivity of membranes probably due to steric obstruction.

FIG. 14 is a diagrammatical illustration of the pH-tunable domain forming lipids that aggregate as the pH of an environment becomes more acidic.

FIG. 15 is a diagrammatical illustration of the pH-tunable domain forming lipids that ‘hide’ or ‘expose’ the surface conjugated targeting ligands depending on the pH of the environment.

FIG. 16 is a graph illustrating the extent of the binding of biotinylated liposomes with 0.01% mole of PEGylated lipids to streptavidin-covered microbeads at various pH levels.

FIG. 17 is a graph illustrating the extent of the binding of biotinylated liposomes with 0.25% mole of PEGylated lipids to streptavidin-covered microbeads at various pH levels.

FIG. 18 is a graph illustrating the extent of the binding of biotinylated liposomes with 0.5% mole of PEGylated lipids to streptavidin-covered microbeads at various pH levels.

FIG. 19 is a graph illustrating the extent of the binding of biotinylated liposomes with 0.75% mole of PEGylated lipids to streptavidin-covered microbeads at various pH levels.

FIG. 20 is a graph illustrating the extent of the binding of biotinylated liposomes with 1.0% mole of PEGylated lipids to streptavidin-covered microbeads at various pH levels.

FIG. 21 is a graph illustrating the extent of the binding of biotinylated liposomes with 1.5% mole of PEGylated lipids to streptavidin-covered microbeads at various pH levels.

FIG. 22 is a thermograph prepared from differential scanning calorimetry data showing the effect of pH on domain formation due to protonation of DSPS lipids.

### DEFINITIONS

Unless specifically indicated otherwise, all technical and scientific terms used herein have the same meaning as commonly understood by those of ordinary skill in the art to which this invention belongs. For purposes of the present invention, the following terms are defined.

The term “cancer” as used herein refers to a disease of inappropriate cell proliferation and is more evident when tumor tissue bulk compromises the function of vital organs. Concepts describing normal tissue growth are applicable to malignant tissue because normal and malignant tissues can share similar growth characteristics, both at the level of the single cell and at the level of the tissue.

The term “anionic liposome” as used herein is intended to encompass any liposome as defined below that is anionic. The liposome is determined as being anionic when present in physiological pH. It should be noted that the liposome itself is the entity that is being determined as anionic. The charge and/or structure of a liposome of the invention present within an in vivo environment has not been precisely determined. However, in accordance with the invention an anionic liposome of the invention will be produced using at least some lipids that are themselves anionic. The liposome need not be comprised completely of anionic lipids but must be comprised of a sufficient amount of anionic lipid such that when the liposome is formed and placed within an in vivo environments at physiological pH the liposome initially has a negative charge.

A “pH-sensitive” lipid as used herein refers to a lipid whose ability to change the net charge on its head group depends at least in part on the pH of the surrounding environment.

“Biologically active agents” as used herein refers to molecules which affect a biological system. These include molecules such as proteins, nucleic acids, therapeutic agents, vitamins and their derivatives, viral fractions, lipopolysaccharides, bacterial fractions, and hormones. Other agents of particular interest are chemotherapeutic agents, which are used in the treatment and management of cancer patients. Such molecules are generally characterized as antiproliferative agents, cytotoxic agents, and immunosuppressive agents and include molecules such as taxol, doxorubicin, daunorubicin, vinca-alkaloids, actinomycin, and etoposide.

“Effective amount” as used herein refers to an amount necessary or sufficient to inhibit undesirable cell growth, e.g., prevent undesirable cell growth or reduce existing cell growth, such as tumor cell growth. An effective amount can vary depending on factors known to those of ordinary skill in the art, which include the type of cell growth, the mode and regimen of administration, the size of the subject, the severity of the cell growth. One of ordinary skill in the art would be able to consider such factors and make the determination regarding the effective amount.

“Liposome” as used herein refers to a closed structure comprising an outer lipid bi- or multi-layer membrane surrounding an internal aqueous space. Liposomes can be used to package any biologically active agent for delivery to cells.

The following abbreviations are used herein: PEG: polyethylene glycol; mPEG: methoxy-terminated polyethylene glycol; Chol: cholesterol; DTPA: diethylenetetramine pentaacetic acid; DPPC: dipalmitoylphosphatidylcholine; DSPA: distearoylphosphatidic acid; and DSPS: distearoylphosphatidylserine.

### DETAILED DESCRIPTION OF PREFERRED EMBODIMENTS

The structural component of a cell membrane is a lipid bilayer. There is increasing evidence that critical cell functions are strongly correlated with reorganization of membranes into lipid rafts although the question whether membrane rafts are functionally relevant is still a controversial one. Lipid rafts are defined as nanometer- to micron-size lipid domains of laterally phase separated lipids. They are suggested to be involved in biological events including membrane trafficking, cell signaling, and viral infection mechanisms. During these events, co-localization of membrane proteins and of other macromolecules occurs on the surface of cells, therefore, changing the effective reactivity and possibly resulting in remodeling of the cell surface topography.

In the present invention, we disclose use of the lipid bilayer membrane as the structural foundation whose lateral reorganization into lipid heterogeneities affects the lateral localization of reactive molecules that are attached to it, with implications in the effective reactivity of the membrane. We use information from the current understanding of molecular interactions that drive lipid phase separation in model membranes, and an external stimulus such as pH to reorganize a model, functionalized lipid bilayer membrane into lipid heterogeneities. However, while the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims.

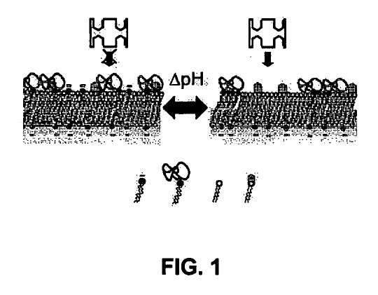

In an illustrative embodiment, using pH as a trigger, this invention provides for the formation of lipid heterogeneities occurring in complex bilayer membranes. FIG. 1 illustrates the tunable surface topography and reactivity of lipid bilayers using pH as a trigger. The upper lipid leaflet represents the outer lipid leaflet of lipid vesicles; the lower lipid leaflet represents the inner lipid leaflet of vesicles. White squares with binding pockets represent streptavidin targets as an example. PEG-lipids and biotin-lipids are also present on the inner lipid leaflet (lower lipid leaflet), and are not shown in FIG. 1 for clarity.

As shown in FIG. 1, the formation of lipid heterogeneities occurring in complex bilayer membranes is shown in the form of vesicles containing (i) a “domain” forming lipid with titratable anionic headgroups (lipid A, darker circles, DSPS), (ii) a lipid A with grafted polymer chains (DSPE-PEG), (iii) non-ionizable lipid B (lighter circles) with hydrocarbon tails that are different from or the same length as those in lipid A (DPPC), and (iv) a lipid with grafted functional groups (5-sided figure) and hydrocarbon tails identical to lipid B (DPPE-biotin). Lowering the pH creates lipid phase separation on the membrane. At high pH values (FIG. 1, left), the lipid A headgroups are charged and repulsion between the headgroups makes the lipid energetically less likely to crystallize. The membrane appears spatially less heterogeneous, and the functional groups are obstructed by surrounding polymer chains. As the pH value is lowered (FIG. 1, right), the anionic A headgroups become protonated, reducing electrostatic repulsion while possibly increasing hydrogen bonding between newly protonated A headgroups. These conditions favor phase separation in which the polymer-conjugated lipids potentially partition into the newly protonated lipid heterogeneities, driven by the dispersive attractive forces between hydrocarbon tails of the same length. In contrast, the functionalized lipids, with hydrocarbon chains identical to the hydrocarbon chains of lipid type B, preferentially partition in the areas with lipids of type B that are depleted in polymer lipids. Therefore, functionalized lipids become exposed and available to interact with their targets (squares containing binding pockets) increasing the effective binding reactivity of membranes.

This process causes reversible reorganization of the bilayer into lipid heterogeneities possibly resulting in remodeling of its surface topography. This process also alters reversibly the membrane's binding reactivity towards its molecular targets. As an example, membrane functionality is introduced by biotinylated lipids. In an illustrative example, the membrane's pH-dependent binding reactivity is evaluated towards streptavidin-covered microparticles as an example for target, within the pH range of 7.4 and 6.7. While streptavidin-covered microparticles are disclosed as an illustrative example, this invention is not limited to streptavidin-covered microparticles as targets. One of ordinary skill in the art will be able to choose other targets without undue experimentation. These pH values correspond to the physiological pH of blood and the interstitial pH of cancerous tumors, respectively. Solid tumors often exhibit a pH gradient between the physiologic pH at perivascular regions and their more acidic core.

This pH range of 7.4 to 6.7 was chosen based on the rationale that these pH-dependent heterogeneous membranes in the form of vesicles may be utilized as targeted drug delivery carriers to advanced vascularized tumors to minimize toxicities and maximize tumor penetration using the following two mechanisms. First, since the molecular targets used for targeted cancer therapy are usually not unique to cancer cells, hiding of the targeting ligands from the vesicle surface during their circulation in the blood (at pH 7.4) may decrease toxicities arising from binding to healthy sites, while exposure of targeting ligands after extravasation in the tumor interstitial space (at pH 6.7) may increase vesicle binding to and uptake by cancer cells. Second, slow diffusion of vesicles in the tumor interstitial space combined with fast internalization rates of antibody-labeled vesicles by tumor cells, fast recycling of targeted antigens, and, fast systemic clearance of vesicles from circulation decreases the penetration depth of these carriers into the tumors. These targeted vesicles with pH-dependent binding reactivity should exhibit increasingly higher reactivity with cancer cells as they diffuse deeper into the more acidic tumor interstitium resulting in greater penetration within the tumor.

Materials and Methods:

Materials. The lipids 1,2-Dipalmitoyl-sn-Glycero-3-Phosphocholine (DPPC), 1,2-Distearoyl-sn-Glycero-3-[Phospho-L-Serine] (Sodium Salt) (DSPS), 1,2-Dipalmitoyl-sn-Glycero-3-Phosphoethanolamine-N-[Methoxy(Polyethylene glycol)-2000] (Ammonium Salt) (DPPE-PEG), 1,2-Distearoyl-sn-Glycero-3-Phosphoethanolamine-N-[Methoxy(Polyethylene glycol)-2000] (Ammonium Salt) (DSPE-PEG), 1,2-Dipalmitoyl-sn-Glycero-3-Phosphoethanolamine-N-(Lissamine Rhodamine B Sulfonyl) (Ammonium Salt) (DPPE-Rhodamine), 1,2-Dipalmitoyl-sn-Glycero-3-Phosphoethanolamine-N-(Biotinyl) (Sodium Salt) (DPPE-biotin lipid), were purchased from Avanti Polar Lipids (Alabaster, Ala.) (all lipids at purity>99%). Dynabeads M 270 Streptavidin (streptavidin coated microparticles) and 1-hexadecanoyl-2-(1-pyrenedecanoyl)-sn-glycero-3-phosphocholine (β-py-C₁₀-HPC) (pyrene-labeled lipid) were obtained from Invitrogen (Carlsbad, Calif.). Calcein, cholesterol, Triton X-100 and phosphate buffered saline (PBS) were purchased from Sigma Aldrich Chemical Company (Milwaukee, Wis.).

Preparation of vesicles. Lipids in chloroform were combined in a 25 mL round bottom flask. Chloroform was evaporated in a Buchi rotavapor R-200 (Buchi, Flawil, Switzerland) for 10 minutes at 55° C. followed by evaporation under N₂ stream for 5 minutes. The dried lipid film was then hydrated in 1 mL of calcein solution (55 mM calcein, 10 mM phosphate buffer, 1 mM EDTA) or of PBS solution (10 mM phosphate buffer, 2.7 mM KCl, 137 mM NaCl, 1 mM EDTA) for 2 hours at 55-60° C. The lipid suspension (10 mM total lipid) was then extruded 21 times through two stacked polycarbonate filters of 100 nm pore diameter (Avestin Inc., Ottawa, Canada). Extrusion was carried out in a water bath at a temperature at least 5° C. higher than the highest Tₘ of the lipids used in each vesicle composition, and vesicle suspensions were incubated for at least 10 minutes at this temperature before initiation of extrusion. After extrusion the vesicle suspension was rapidly cooled to room temperature, by letting the suspension for one-half hour to reach room temperature. To remove the unentrapped calcein and to exchange the vesicle surrounding solution with isosmolar PBS of different pH values (7.4, 7.0, 6.7, 6.5), the vesicle suspension was then divided into four equal volumes. Each volume was eluted at room temperature through an 11 cm Sephadex G-50 size exclusion chromatography (SEC) column, and vesicles were then transferred to an incubator at 37° C. for further measurements.

Dynamic Light Scattering. The size distributions of vesicle suspensions were measured by dynamic light scattering (DLS), twenty-four hours after extrusion followed by incubation of vesicles at 37° C., using an N4 Plus autocorrelator (Beckman-Coulter) equipped with a 632.8 nm He—Ne laser light source. The measurement protocol is published in Sofou, S., et al., J. Nucl. Med., 45, 253-260 (2004), which is incorporated herein.

Differential Scanning Calorimetry. A VP-DSC Instrument (MicroCal, LLC, Northampton, Mass.) was used for the differential scanning calorimetry (DSC) studies. DSC scans were performed on vesicle suspensions of 0.5 mL sample volume containing 2.5 mM total lipid. Vesicles were prepared having both lipid leaflets exposed to the same pH value. The corresponding PBS buffer (pH 7.4, 7.0, 6.7 and 6.5) was introduced to the dried lipid. The thermograms of vesicle suspensions were acquired from 20° C. to 85° C. at a scan rate of 5° C./hr. Scans were acquired twenty-four hours after extrusion of vesicles followed by incubation of vesicles at 37° C. The excess heat capacity curves were normalized by subtraction of the thermograms of the corresponding buffers that were acquired at identical conditions.

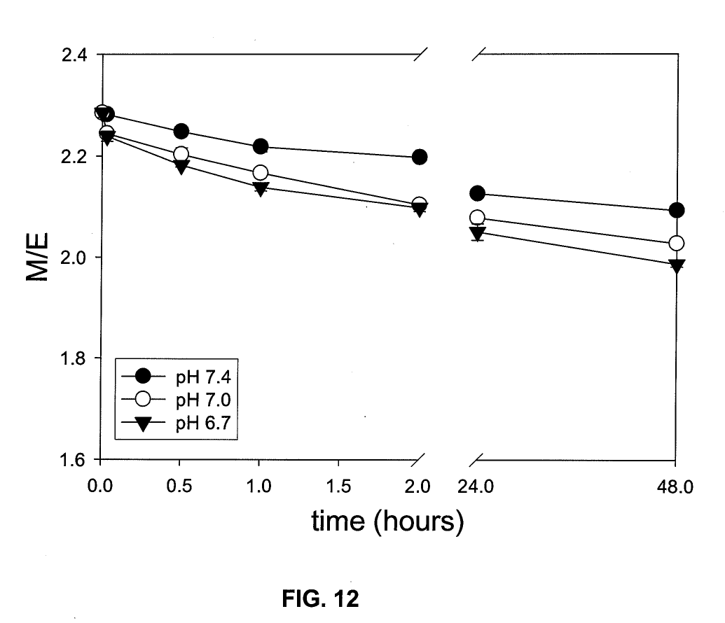

Binding measurements. Biotinylated vesicles containing self quenching concentrations of calcein solution (55 mM calcein, 10 mM phosphate buffer, 1 mM EDTA, pH=7.4) and rhodamine-labeled lipids were introduced to their targets after vesicle incubation for twenty-four hours at the corresponding pH values at 37° C. to minimize interference in binding from effects related to the kinetics of formation of heterogeneities (see FIG. 12). Vesicles were then incubated with streptavidin-functionalized magnetic microparticles (6.09×10⁷ microbeads/mL in 1.1 mL of 227 μM lipid) at 37° C. for twenty-four hours at different pH values. Bound vesicles were separated from unbound vesicles using magnetic separation followed by ten washing steps. Fluorescence intensities of rhodamine-labeled lipids (ex: 550 nm, em: 590 nm), and of calcein (ex: 495 nm, em: 515 nm) were measured after addition of Triton-X 100 that causes release of encapsulated calcein from bound vesicles and solubilizes the lipids of bound vesicles.

Referring to FIG. 12, to minimize interference in binding from effects related to the kinetics of formation of heterogeneities, the time required by the bilayers to reach a state not significantly altered over time after pH change was estimated. Vesicles composed of equimolar ratios of DPPC and DSPS lipids, 5% mol cholesterol, 0.5% mol DSPE-PEG lipids and pyrene-labeled lipids were introduced to different transmembrane pH gradients at time t=0. The M/E ratio was monitored for three days.

Lipid membranes were prepared at temperatures where all lipids are in the fluid phase followed by fast cooling at 37° C. But, even at 37° C., the lateral diffusion coefficient of DPPC lipids (Tₘ=41° C.) has been reported to be 10⁻¹⁰ cm²/s: two orders of magnitude slower compared to the fluid state. Where pH gradients are introduced at 37° C., the lipid mixtures are composed of equimolar fractions of DPPC and DSPS lipids (Tₘ=68° C.). Even at the highest pH studied (7.4), DPPC-rich areas exist, and in these areas the lipids are expected to diffuse slowly. This low fluidity is expected to affect the rate of diffusion of newly protonated DSPS lipids and to slow down the rate of formation of DSPS-rich phase-separated domains on the plane of the membrane.

However, given the short distances—on the surface of a 100 nm in diameter vesicle—that are required to be traveled by lipids in order to result in domain formation, incubation of the bilayer at 37° C. for several hours appears to be adequate to reach a state not significantly altered over time. In particular, to obtain an estimate of this time, the changes in the phenomenological lipid mobility upon pH change using the pyrene-labeled lipid technique were monitored over three days. The data suggest that 24 hours at 37° C. after decrease of the suspension's pH is adequate time for the bilayer to reach a state that does not seem to significantly change afterwards over time. Contrary to studies where suspension pH is lowered at 37° C., all DSC studies are performed on bilayers that were introduced to the indicated pH conditions when lipids were in the fluid phase to minimize the effect of the kinetic component.

Detection of monomer-to-excimer emission shift vs. pH. Unilamellar vesicles (15 μM lipid) were prepared in PBS at pH 7.4 (10 mM phosphate buffer, 2.7 mM KCl, 137 mM NaCl, 1 mM EDTA) in the absence of transmembrane pH gradients. At t=0, a pH gradient was introduced across the bilayer by lowering the suspension's pH value, and the monomer (M) (ex: 344 nm, em: 396 nm) and excimer (E) (ex: 344 nm, em: 470 nm) fluorescence intensities were monitored at 37° C. Two hours later, the pH of the outer lipid leaflets was raised back to 7.4 followed by reintroduction of transmembrane pH gradients two or four hours later.

Results:

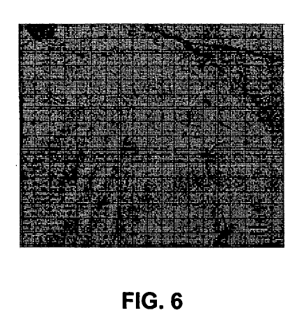

Vesicle lamellarity and size distributions. All studies were performed on lipid membranes in the form of unilamellar vesicles composed of lipids with non-matching acyl-tail lengths, dipalmitoylphosphatidyl choline (DPPC) and distearoylphosphatidyl serine (DSPS), and cholesterol (See FIG. 6). The average sizes of vesicles with variable contents of distearoyl phosphoethanolamine-PEG (DSPE-PEG) (0.1%, 0.5%, and 1.5% mol) that were measured by DLS ranged from 107±5 nm to 120±12 nm in diameter. The average values measured in solution were in general agreement with the sizes observed in cryo-TEM samples. FIG. 6 is a Cryo-TEM image showing unilamellar vesicles composed of equimolar DPPC and DSPS lipids with 5% mol cholesterol, and 0.5% mol DSPE-PEG lipids (scale bar is 100 nm). Vesicles were frozen, and thin frozen sections were imaged without staining using a FEI Tecnai 20 cryo-transmission electron microscope.

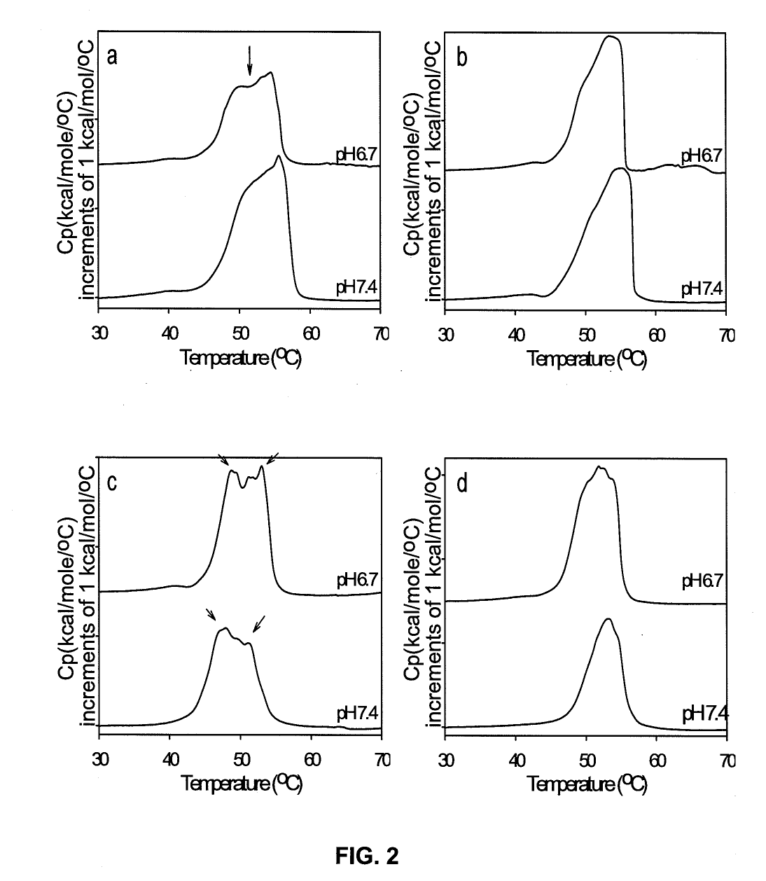

Cholesterol affects the pH dependence of heterogeneities. The effect of cholesterol on the pH dependence of formation of heterogeneities was studied on vesicles containing equimolar ratios of DPPC lipid (Tₘ=41° C.) and DSPS lipid (Tₘ=68° C.) at the pH values of 7.4 and 6.7. FIG. 2a shows that in bilayers containing 5% mol cholesterol, lipid heterogeneities with distinct lipid packing properties are formed at the lower pH of 6.7. This is suggested by the clear split on the main thermal transition at pH 6.7 indicated by the vertical arrow. The observed pH-dependent response is attributed to the protonation of the carboxyl group of phosphatidylserine headgroups that possibly results in attractive interactions between the headgroups of DSPS lipids via hydrogen bonding, forming, therefore, heterogeneous domains rich in newly protonated DSPS lipids. Attractive Van der Waals interactions among the matching acyl-tail lengths of the newly protonated DSPS lipid could also favorably contribute to the observed response in membrane reorganization that occurred when all lipids were in the fluid state during sample preparation. It is not clear by our measurements if cholesterol would exhibit preferential partition in DPPC-rich or in DSPS-rich domains, because the miscibility of cholesterol with phospholipid membranes depends on the interactions between the hydrocarbon chains of phospholipids and the attractions between their headgroups. In phase separated membranes these interactions vary within each lipid domain, and the partition of cholesterol in each domain can only be known if the exact composition of the domain is also known. In mixtures of two types of phosphatidylcholines with different acyl-tail lengths, exhibiting different fluidity and therefore different miscibility with cholesterol, addition of cholesterol has been shown to induce ordered packing among one type of lipids resulting in formation of lateral phase separated domains. In illustrative embodiments of the present invention, in an effort to assign pH dependence to the formation of membrane heterogeneities, the headgroups of the two lipid types are also different, with one being titratable within the pH range of interest.

FIG. 2 illustrates how cholesterol and the PEGylated lipid's acyl-tail length affect the extent of formation of membrane heterogeneities with pH. DSC scans of vesicles containing equimolar ratios of DPPC lipid (with gel-liquid transition temperature Tₘ=41° C.) and DSPS lipid (Tₘ=68° C.) at the pH values of 7.4 and 6.7. FIG. 2a shows bilayers containing 5% mol cholesterol. FIG. 2b shows bilayers not containing cholesterol. FIG. 2c shows bilayers containing 5% mol cholesterol and 0.5% mol DSPE-PEG lipids. FIG. 2d shows bilayers containing 5% mol cholesterol and 0.5% mol DPPE-PEG lipids. DSC scans from 25° C. to 85° C. at a scan rate of 5° C./hr were performed on vesicle suspensions of 0.5 mL sample volume containing 2.5 mM total lipid.

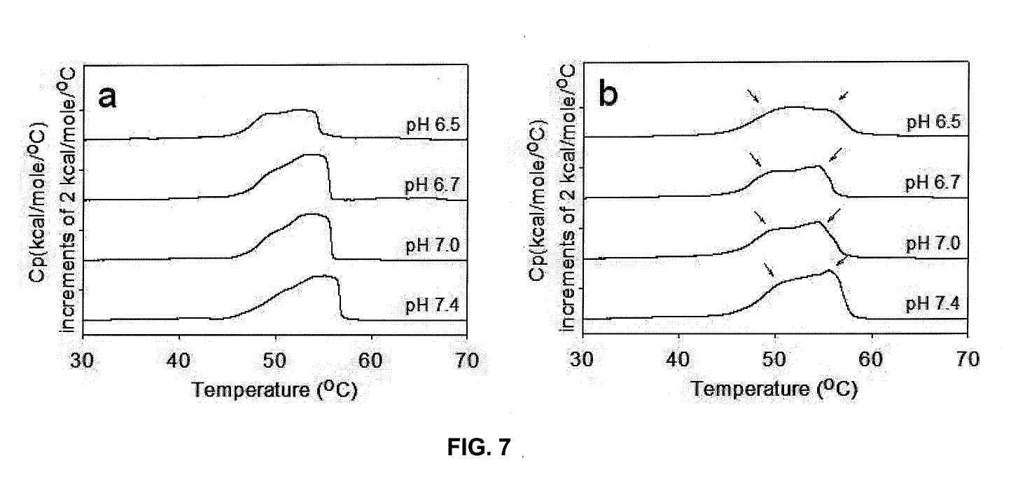

In the absence of cholesterol, FIG. 2b shows that bilayers composed of equimolar DPPC and DSPS lipids, exhibit thermal responses with unresolved thermal peaks, and a monotonic shift of the thermal spectrum by 1.3 degrees towards lower temperatures with decreasing pH from 7.4 to 6.7. The thermal shift increases to 2.3 degrees from pH 7.4 to 6.5 (see FIG. 7a). Comparison of thermal spectra at intermediate pH values within the pH range of 7.4 and 6.5 shows that cholesterol plays a pivotal role in promoting pH dependence on the extent of lipid heterogeneities in membranes containing DPPC and DSPS lipids, therefore, affecting their collective behavior within the bilayer (see FIGS. 7a and 7b for thermal spectra obtained at intermediate pH values).

Referring to FIG. 7, DSC scans of vesicles containing equimolar ratios of DPPC lipid (Tₘ=41° C.) and DSPS lipid (Tₘ=68° C.) at the pH range from 7.4 to 6.5. FIG. 7a shows bilayers not containing cholesterol. FIG. 7B shows bilayers containing 5% mol cholesterol. Two major thermal transitions at approximately T₁=47° C. and T₂=55° C. are indicated by arrows. The ratios of thermal transition intensities at T₂ over T₁ exhibit strong pH dependence as shown on Table 1:

TABLE 1
Cp_{, T2}/Cp_{, T1}*

| pH | 5% mol cholesterol |
|---|---|
| 7.4 | 4.20 |
| 7.0 | 1.27 |
| 6.7 | 1.12 |
| 6.5 | 1.10 |

*DSC scans from 25° C. to 85° C. at a scan rate of 5° C./hr were performed on vesicle suspensions of 0.5 mL sample volume containing 2.5 mM total lipid.

The membrane composition of equimolar DPPC and DSPS lipids containing cholesterol, shown in FIG. 2a, is used in the present invention as the “structural substrate” that may exhibit pH-triggered surface patterning, due to formation of lipid heterogeneities. One aim of the present invention is to translate the pH-dependent two-dimensional surface patterning of the bilayer into a pH-dependent three-dimensional architecture that extends beyond the surface of the membrane. To achieve this, included in the bilayer are lipids with headgroups modified by bulky PEG chains. These lipids are chosen to have acyl-tail lengths that match either the dipalmitoyl or the distearoyl acyl-tail lengths of the DPPC or DSPS lipids, respectively. Preferential partition of PEGylated lipids into the newly formed lipid heterogeneities with lowering of the pH would result in polymer-enriched and polymer-depleted areas, therefore, changing the membrane surface topography.

PEGylated lipid's acyl-tail length affects the formation of membrane heterogeneities. In bilayers containing 5% mol cholesterol, comparison between membranes not containing PEGylated lipids (FIG. 2a) and membranes containing 0.5% mol DSPE-PEG lipids (FIG. 2c), shows that PEGylated lipids with acyl-tail lengths matching the acyl-tails of the titratable DSPS lipid promotes formation of distinct multipeak thermal responses. In addition, a pronounced split is observed on the major thermal transition at the lower pH value of 6.7. On the contrary, substitution of the type of PEGylated lipid with 0.5% mol DPPE-PEG lipids, with acyl-tails matching the lengths of the DPPC lipid, abolishes the presence of distinct thermal transitions, and results in less pronounced pH-dependent thermal responses as observed by DSC (FIG. 2d). The observed enhancement of formation of membrane heterogeneities with addition of DSPE-PEG in membranes containing cholesterol could be attributed to favorable Van der Waals attractions among the matching distearoyl acyl-tails of DSPE-PEG lipids and DSPS lipids.

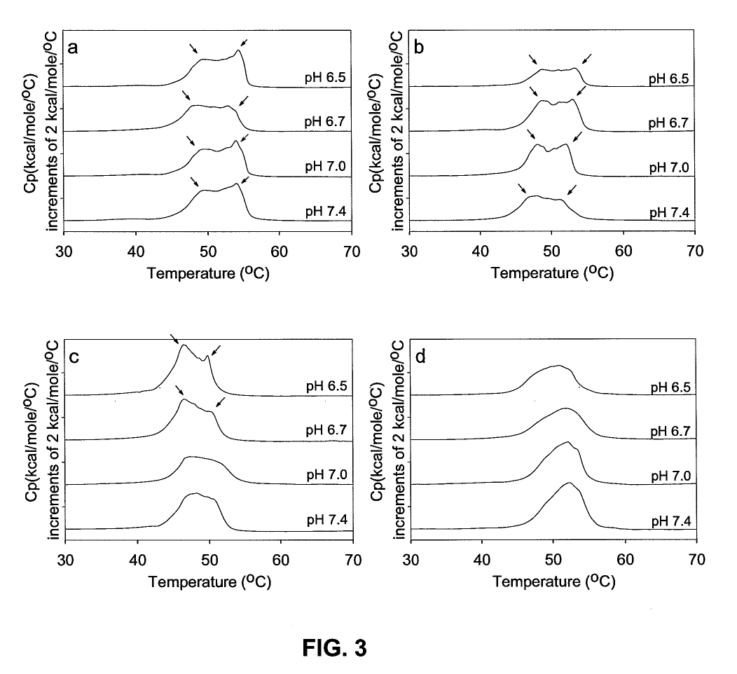

DSPE-PEG lipids content affects the extent of formation of membrane heterogeneities and their pH dependence. At relatively low contents of 0.1% mol and 0.5% mol of DSPE-PEG lipid, shown in FIG. 3a and FIG. 3b, respectively, unilamellar vesicles composed of equimolar ratios of DPPC and DSPS lipids with 5% mol cholesterol, exhibit multipeak thermal spectra at all pH values ranging from 7.4 to 6.5. This response is suggestive of the presence of heterogeneous membranes with several distinct lipid phases. Two major thermal transitions at approximately T₁=47° C. and T₂=55° C. are indicated by arrows. The ratios of thermal transition intensities at T₂ over T₁ exhibit strong pH dependence, and, with one exception at pH 6.7 of 0.1% mol, increase with decreasing pH (see Table 2, which shows DSPE-PEG lipid's content affects the extent of formation of membrane heterogeneities and their pH dependence). This response suggests that with decreasing pH, increasing formation of lipid phases occurs that are rich in possibly the higher Tₘ lipid component, the newly protonated DSPS lipid. These phases are possibly stabilized by intermolecular hydrogen bonding between the protonated amino groups of the phosphatidylserine lipids and deprotonated phosphate groups of their headgroups, and by Van der Waals attractions among matching acyl-tail lipids. These heterogeneities possibly contain preferentially associated DSPE-PEG lipid as discussed above that would alter the surface topography of the membrane. Experimental indications supporting this suggestion are presented by the change of the membrane's effective binding reactivity (vide infra).

TABLE 2**

| | Cp_{, T2}/Cp_{, T1} | Cp_{, T2}/Cp_{, T1} |
|---|---|---|
| | 0.1% mole | 0.5% mol |
| pH | DSPE-PEG | DSPE-PEG |
| 7.4 | 1.22 | 0.85 |
| 7.0 | 1.31 | 1.02 |
| 6.7 | 0.95 | 1.02 |
| 6.5 | 1.32 | 2.44 |

**Table 2 provides ratios of thermal transition intensities of vesicles at approximately T₂ = 55° C. over T₁ = 47° C. versus pH for 0.1% mol and 0.5% mol DSPE-PEG lipid contents of membranes composed of equimolar DPPC and DSPS lipids with 5% mol cholesterol as shown in FIGS. 3a and 3b. Contributions from relative thermal transitions at the higher temperature, T₂, are increasing with decreasing pH.

FIG. 3 illustrates DSPE-PEG lipid's content affects the extent of formation of membrane heterogeneities and their pH dependence. DSC scans of vesicles containing equimolar ratios of DPPC lipid and DSPS lipid with 5% mol cholesterol and variable fractions of DSPE-PEG lipid at pH values ranging from 7.4 to 6.5. DSPE-PEG content: FIG. 3a 0.1% mol, FIG. 3b 0.5% mol, FIG. 3c 1.0% mol, and FIG. 3d 1.5% mol.

Further increase in the fraction of DSPE-PEG lipid to 1.0% mol results in significant loss of multipeak thermal contributions except for the lowest studied pH values of 6.7 and 6.5 (FIG. 3c). Increase of DSPE-PEG lipid to 1.5% mol eliminates all distinct multipeak thermal contributions (FIG. 3d). Instead, a broad thermal transition is formed that exhibits width broadening with decreasing pH. Undulation of the grafted polymer chains acting against lipid order in the underlying bilayer could be the main contributing factor for the observed loss of formation of pH-dependent heterogeneities.

Formation of heterogeneities is reversible with pH. To evaluate in real time the reversibility of lipid heterogeneities with respect to pH, the changes of the monomer-to-excimer emission shift (M/E) upon repetitive changes of the outer lipid leaflets' pH in bilayers of unilamellar vesicles containing pyrene-labeled lipids were monitored. Lipids are labeled with pyrene at the free end of one of their acyl tails, and due to the bulky pyrene-group, pyrene-labeled lipids are expected to partition at relatively lower extents into more well packed membrane domains. Consequently, increasing formation of lipid heterogeneities alters the lateral distances among pyrene-labeled lipids demonstrated by a decrease in the M/E ratio. The observed rates of change in the M/E ratios should be indicative of lipid mobilities during the pH-dependent lipid separation process affecting the kinetics of formation of membrane heterogeneities. At the working temperature of 37° C., the DSC scans indicate that these studies are performed on gel phase membranes. Lipid membranes are composed of equimolar fractions of DPPC and DSPS lipids. At 37° C., the lateral diffusion coefficient of DPPC lipids (Tₘ=41° C.) has been reported to be 10⁻¹⁰ cm²/s: two orders of magnitude slower compared to 5×10⁻⁸ cm²/s in the fluid state.

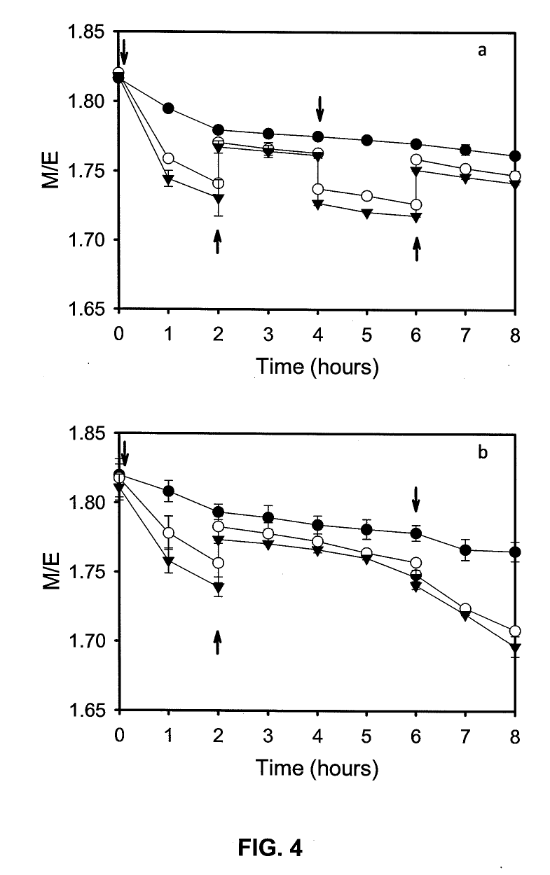

FIG. 4a shows the M/E ratio as the vesicle suspension's pH value is decreased at t=0 and t=4 hours (indicated by the arrows pointing down). The M/E ratio decreases proportionally to the deviation in the pH value of the vesicle suspension from the initial pH value of 7.4 (7.4-7.0<7.4-6.7<7.4-5.5). Neutral (7.4) is also the pH of the encapsulated aqueous volume of vesicles. The lower pH value corresponds to the pH of solution facing the outer lipid leaflet. Great extent of reversibility is observed for the M/E ratios upon increase of the vesicle suspension's pH back to the initial value of 7.4 at t=2 and t=6 hours (indicated by the arrows pointing up). Significant recovery of the M/E ratios to values close to the reference values of no transmembrane pH gradient (indicated by the filled circles in FIG. 4a) suggests extensive reversibility in the formation of lipid heterogeneities with respect to pH. Lipid bilayers composed of phosphatidylcholine lipids and not containing titratable phosphatidylserine lipids did not exhibit changes in the M/E ratios upon introduction of transmembrane pH gradients within the range of interest (from pH 7.4 to 6.5) (data not shown).

FIG. 4 illustrates the reversibility of the M/E ratio upon repeated introduction and removal of transmembrane pH gradients across the membranes of unilamellar vesicles suggests reversibility in the formation of lipid heterogeneities. Collective lipid mobilities during formation of pH-induced lipid heterogeneities are monitored by the monomer-to-excimer (M/E) emission shift of pyrene-labeled lipids. Vesicles composed of equimolar DPPC and DSPS lipids with 5% mol cholesterol and 0.5% mol DSPE-PEG lipids were prepared in PBS at pH 7.4 in the absence of any transmembrane pH gradients (). At t=0 the pH of the outer lipid leaflet of vesicles was dropped to pH=6.7 (∘) or 5.5 (▾) by decreasing the vesicle suspension's pH (indicated by arrows pointing down). At later times the vesicle suspension's pH was increased back to the initial value of 7.4 (indicated by arrows pointing up). The process of decreasing the vesicle suspension's pH was then repeated after two hours (FIG. 4a) and after four hours (FIG. 4b). Error bars correspond to standard deviations of repeated measurements (two vesicle preparations, three samples per preparation per time point). The plotted M/E ratios were recorded within three minutes after pH change. Vesicles were prepared containing 4% mol pyrene-labeled lipids.

It is noteworthy that at the first time of lowering the pH at t=0 in FIG. 4a, the initial rates of decrease of M/E are slower than the initial rates of M/E decrease measured upon removal and reintroduction of the same pH gradients at t=4 hours. At t=4 hours, the initial rates of M/E decrease appear almost instantaneous. When a longer “relaxation” time at pH 7.4 of four hours instead of two hours is given to the bilayer before repeating the introduction of low pH on the outer lipid leaflet as shown in FIG. 4b, at t=6 hours, then the observed initial rates of M/E decrease are comparable to the rates measured at the first time of lowering the pH at t=0. This response suggests a memory property of the membrane (at the time points when pH change is indicated by arrows, the M/E ratios were measured immediately before pH change and within three minutes after pH change).

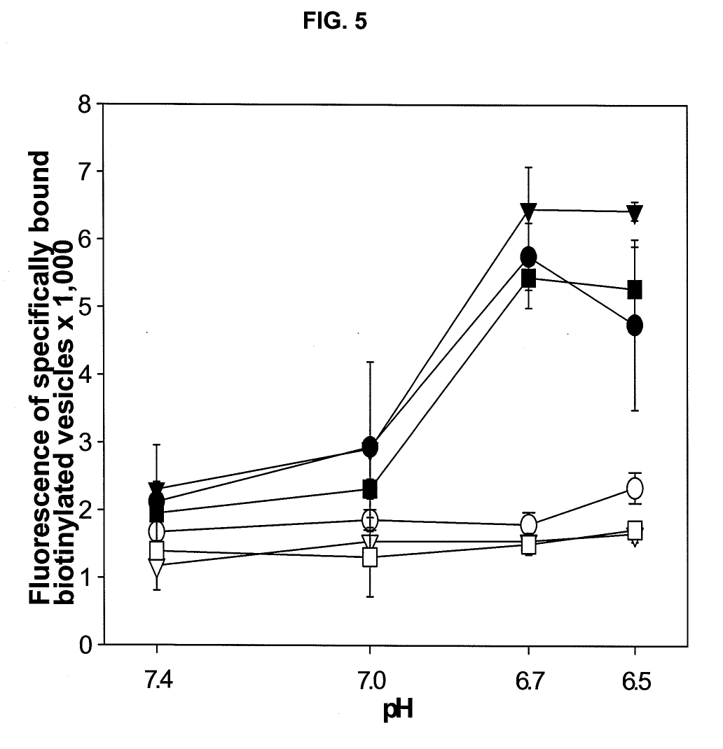

Binding reactivity is correlated with pH-dependent formation of lipid heterogeneities. FIG. 5 shows that the extent of specifically bound biotinylated vesicles depends on the vesicle suspension's pH and on the heterogeneous membrane's content in DSPE-PEG lipid. In particular, for DSPE-PEG contents ranging from 0.25% to 0.75% mol (closed symbols in FIG. 5), vesicles exhibit strong pH-dependent binding that increases by approximately 177% between pH 7.4 and 6.7.

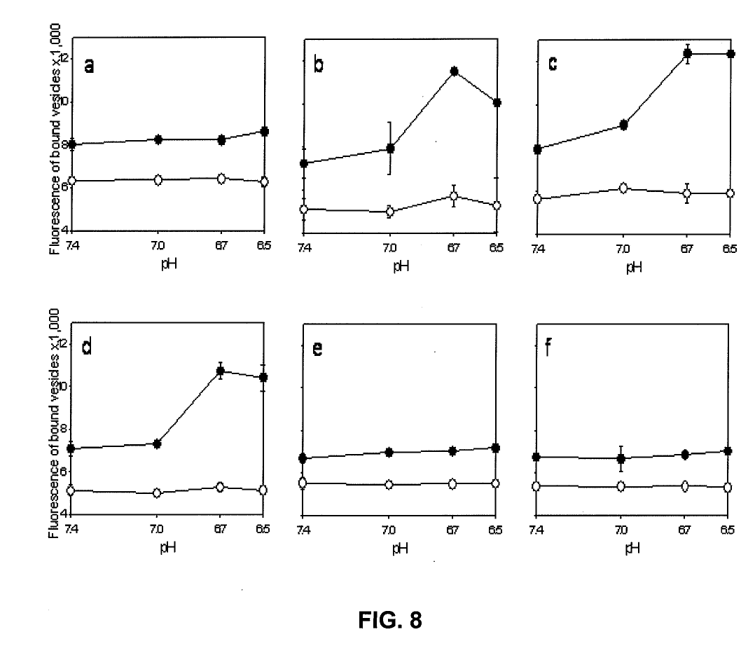

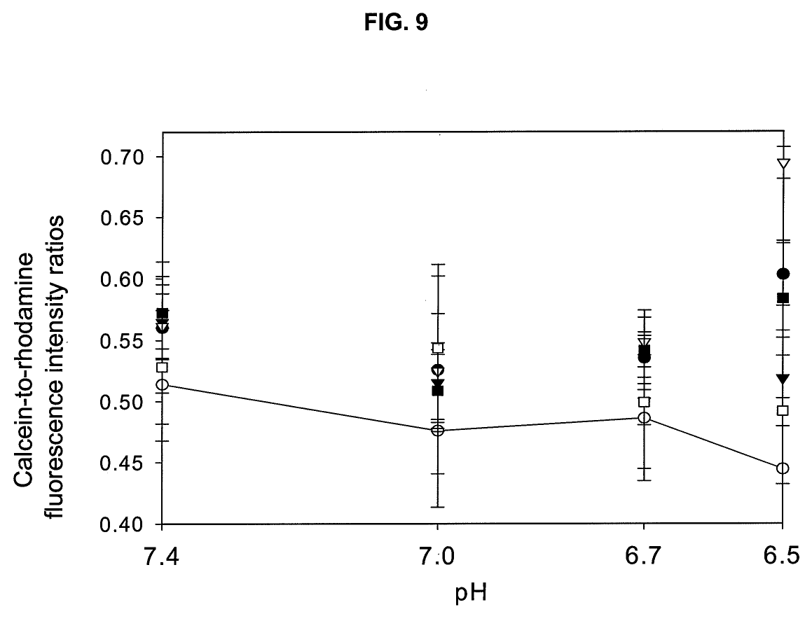

FIG. 5 shows that at concentrations of DSPE-PEG lipid lower than 0.25% mol or higher than 0.75% mol, biotinylated vesicles exhibit pH-independent binding (open symbols). At 0.10% mol of DSPE-PEG lipid, the extent of biotinylated vesicles that is associated with the streptavidin-covered microparticles is moderately higher than that of non-biotinylated vesicles (see FIG. 8a). However, these biotinylated vesicles also exhibit the lowest encapsulated content-to-lipid ratios of all bound biotinylated vesicles evaluated (see FIG. 9), suggesting strong vesicle adsorption possibly via multipoint contacts leading to vesicle deformation and content leakage. At 1.0% mol of DSPE-PEG lipid or higher, biotinylated vesicles exhibit pH-independent binding that is only fairly greater than the binding of non-biotinylated vesicles (see FIG. 8e). pH-independent binding at these higher contents of PEGylated lipids coincides with loss of pH-dependence of the extent of formation of lipid heterogeneities observed by DSC (FIG. 3d).

Referring to FIG. 8, fluorescence intensities of lipids of bound biotinylated vesicles to streptavidin-coated microparticles (closed symbols), and of bound non-biotinylated vesicles to streptavidin-coated microparticles (open symbols). Vesicles were composed of equimolar ratios of DPPC and DSPS lipids, 5% mol cholesterol and different contents of DSPE-PEG lipids: FIG. 8a shows 0.10% mol, FIG. 8b shows 0.25% mol, FIG. 8c shows 0.50% mol, FIG. 8d shows 0.75% mol, FIG. 8e shows 1.00% mol, and FIG. 8f shows 1.50% mol. Error bars correspond to standard deviations of repeated measurements (three vesicle preparations, two samples per preparation per pH point). The solid lines connecting the measured intensities are used as guide to the eye. Vesicles were labeled with 1.0% mol DPPE-Biotin and 0.5-1.0% mol DPPE-Rhodamine lipids.

Referring to FIG. 9, content release from vesicles upon binding suggesting deformation of bound vesicles, was evaluated at different surface grafting densities of PEG-labeled lipids vs. pH. The ratios of content-to-lipid of bound vesicles were measured by comparing the intensities of encapsulated fluorophores to lipid-conjugated fluorophores of bound vesicles. For both biotinylated and non-biotinylated vesicles the content-to-lipid ratios were not a strong function of pH. For all PEG-grafting densities studied, content-to-lipid ratios were higher for the non-biotinylated vesicles than for biotinylated vesicles.

Multipoint contacts per bound vesicle between biotinylated lipids and surface immobilized streptavidin could cause vesicle deformation resulting in content release. The vesicles with the lowest content of 0.1% mol DSPE-PEG exhibited consistently lower content-to-lipid ratios compared to all vesicles studied.

Vesicles containing self-quenching concentrations of calcein (55 mM, pH=7.4) and rhodamine-labeled lipids were incubated at 227 μM final total lipid concentration with streptavidin-functionalized magnetic microparticles (6.09×10⁷ microparticles/mL of final solution in 1.1 mL of total incubation suspension) at 37° C. for 24 hours at the corresponding pH values. Bound vesicles were separated from unbound vesicles using a Dynal Magnetic Particle Concentrator according to manufacturer's instructions followed by ten washing steps with PBS (pH=7.4). After separation, Triton-X 100 was added to the mixture of bound vesicles and magnetic microparticles to solubilize the lipids of bound vesicles and to release the calcein encapsulated in bound vesicles. Biotinylated vesicles were composed of equimolar ratios of DPPC and DSPS lipids, 5% mol cholesterol and DSPE-PEG lipids at different contents: 0.10% mol (∘), 0.25% mol (), 0.50% mol (▾), 0.75% mol (▪), 1.00% mol (∇), 1.50% mol (□). The solid line connecting the measured ratios for the lowest membrane coverage is used as guide to the eye. Error bars correspond to standard deviations of repeated measurements (three vesicle preparations, two samples per preparation per pH point).

FIG. 5 illustrates that pH-dependent vesicle binding reactivity is correlated with pH-dependent formation of lipid heterogeneities. Biotinylated vesicles, as an illustrative example of functionalized liposomes, composed of equimolar DPPC and DSPS lipids with 5% mol cholesterol that contain PEGylated lipids with acyl-tails matching the length of the phosphatidylserine lipids, exhibit tunable target recognition vs. pH that is controlled by the formation of free-of-PEG open spaces on the membrane surface for receptor docking to occur between surface grafted PEGs. Biotinylated vesicles were incubated with streptavidin-coated magnetic microparticles, which are used herein as an example of targeted cells. The total microparticle surface area was chosen to be two orders of magnitude larger than the total lipid bilayer area. DSPE-PEG contents: 0.10% mol (∘), 0.25% mol (), 0.50% mol (▾), 0.75% mol (▪), 1.00% mol (∇), 1.50% mol (□). The extent of specifically bound biotinylated vesicles corresponds to the measured fluorescence intensities of lipids of bound biotinylated vesicles to streptavidin-coated magnetic microparticles that were corrected for the measured fluorescence intensities of lipids of bound non-biotinylated vesicles of identical lipid compositions. Error bars correspond to standard deviations of repeated measurements (three vesicle preparations, two samples per preparation per pH point). The solid lines connecting the measured intensities are used as a guide to the eye. Vesicles were labeled with 1% mol DPPE-Biotin and 0.5-1.0% mol DPPE-Rhodamine lipids.

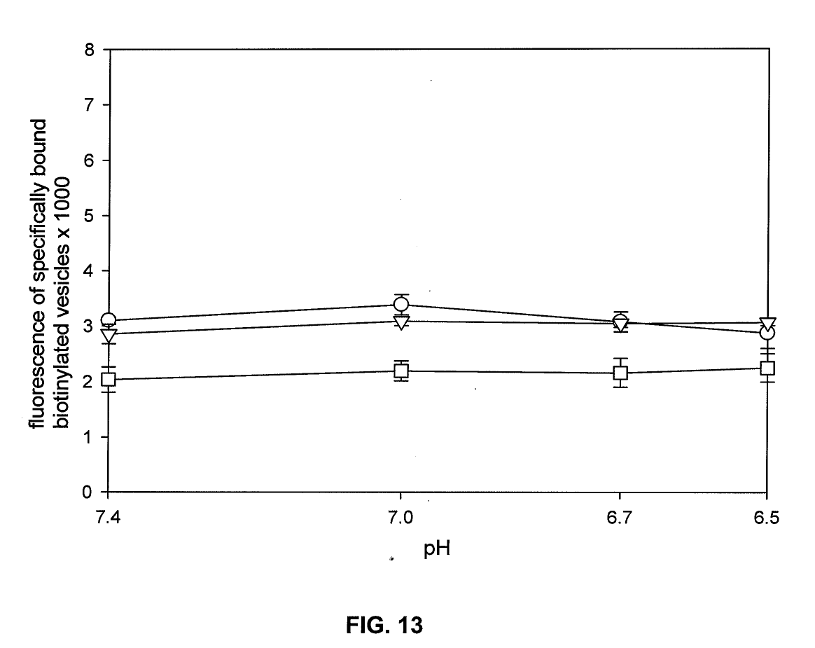

To demonstrate that the observed change in binding reactivity vs. pH is due to change in the fraction of exposed biotins on the vesicle surface and not due to possible changes in the configuration of the PEG-lipid which could itself alter the binding reactivity, vesicles without phosphatidylserine lipids were evaluated. In particular, vesicles of uniform membranes composed of DPPC lipids containing DPPE-biotin and different fractions of DPPE-PEG (0%, 0.5%, 1.5% mol) corresponding to different extents of surface coverage by PEG-chains, exhibit: (a) pH-independent binding to streptavidin-covered microparticles, and (b) decreasing extents of specific binding with increasing contents of DPPE-PEG that is attributed to increasing steric repulsion mediated by the PEG-chains (see FIG. 13).

Referring to FIG. 13, biotinylated vesicles composed of DPPC lipids with 5% mol cholesterol that contain DPPE-PEGylated lipids with acyl-tails matching the length of the phosphatidylcholine lipids, do not exhibit tunable target recognition vs. pH. These membranes do not exhibit pH-dependent changes in their DSC thermograms within the pH range of 7.4 and 6.5. Biotinylated vesicles were incubated with streptavidin-coated magnetic microparticles. The total microparticle surface area was chosen to be two orders of magnitude larger than the total lipid bilayer area. DPPE-PEG contents: 0% mol (∘), 0.5% mol (∇), 1.50% mol (□). The extent of specifically bound biotinylated vesicles corresponds to the measured fluorescence intensities of lipids of bound biotinylated vesicles to streptavidin-coated magnetic microparticles that were corrected for the measured fluorescence intensities of lipids of bound non-biotinylated vesicles of identical lipid compositions. The solid lines connecting the measured intensities are used as guide to the eye. Vesicles were labeled with 1% mol DPPE-Biotin and 1.0% mol DPPE-Rhodamine lipids.

DSC studies suggest that DPPC/DSPS/cholesterol bilayer mixtures containing PEGylated lipids with acyl-tails matching the length of the phosphatidylserine lipids exhibit formation of pH-dependent heterogeneities. DSPE-PEG lipids may associate at different extents with the different heterogeneities, therefore, modifying in different ways the membrane surface topography within 3 to 4 nm s from the bilayer surface in a pH-dependent manner. At the limit of higher lipid mixing within the bilayer (occurring at higher pH values), membrane-conjugated functional groups would become sterically hindered towards binding to their targets due to the presence of adjacently grafted PEG chains (FIG. 1, left). When higher extents of formation of heterogeneities take place (lower pH) forming areas on the membrane surface depleted from grafted PEG chains (FIG. 1, right), then the functional groups would become available towards binding. In this invention, to attribute functionality to the lipid membrane, the small binding group biotin was chosen to be directly conjugated on the lipid headgroups, and reactivity of biotinylated vesicles versus pH was evaluated towards binding to streptavidin-coated magnetic microparticles.

Specific vesicle binding due to biotin-streptavidin recognition should occur when the vesicle topography, determined by the grafted PEG chains, exhibits transient “open” spaces on the membrane surface that are free of PEG chains (FIG. 1, right) and adequately large to accommodate docking of one streptavidin molecule (5.4×5.8×4.8 nm³). The average distance on the plane of the membrane between DPPE-biotin lipids (1% mol) is relatively short (6.9 nm) and comparable to the size of streptavidin, assuming homogeneous distribution on the vesicle surface and an area A per lipid molecule in the bilayer equal to 48 Å² for gel phase membranes. When increasing extents of lipid heterogeneities occur with lowering pH, this distance could locally be shorter than the above value. The effective distances (D_{eff}) on the surface of the membrane that define the dimensions of these transient free-of-PEG open spaces should equal to D−2*R_{f}, where D is the distance between PEG lipids on the plane of the bilayer (=(A/M)^{1/2}, and M is the mole fraction of PEG lipid), and R_{f}=N^{3/5}*a=3.8 nm is the radius of half-sphere approximately occupied by each PEG chain in the mushroom regime that is also shorter than all three dimensions of streptavidin. It is not possible to calculate the effective distances D_{eff} without knowing the exact extent of phase separation and the membrane topography. However, the estimated effective distances assuming uniform bilayers for the studied PEG-grafting densities are comparable with the dimensions of streptavidin suggesting that, at least qualitatively, the observed extents of formation of lipid heterogeneities would result in increase of D_{eff} to values greater than the dimensions of streptavidin allowing its docking leading to vesicle binding.

Potential dissociation of PEGylated lipids from the bilayer with decreasing pH does not seem to be a possible alternative mechanism for the suggested formation of transient free-of-PEG open spaces on the surface of the membrane: vesicles retain the pH-dependent binding response after repeated cycles of altering the vesicle's suspension pH between the values of 7.4 and 6.5 (Table 3). In addition, no change was observed on the average vesicle size due to repeated changes of the pH value of the vesicle suspension (data not shown). During formation of heterogeneities, the local grafting density of PEGylated lipids, in areas enriched in PEGylated lipids, would also increase. However, given the observed reversibility on the binding reactivity of the membranes, this higher local density of PEGylated lipids is not expected to exceed the critical value of approximately 10% mol (for 2000 molecular weight of PEG) that could irreversibly result in structures other than lamellae, such as micelles. Increase of lipid heterogeneities with lowering pH and formation of DPPC-rich and DSPS-rich domains may result in membrane phases of variable rigidity at 37° C., and most importantly, in boundaries between the phases that may give rise to high line tension. Minimization of the boundary energy by decreasing the boundary perimeter could result in (positive or negative) bulged areas. This response would not necessarily contradict our suggestion of pH-dependent formation of transient free-of-PEG open spaces on the lipid membrane required for the observed binding to take place, since enrichment or depletion of budded areas with grafted PEGylated lipids would still result in formation of transient free-of-PEG open spaces on the curved membrane.

TABLE 3***
Functionalized vesicles exhibit reversible binding reactivity with pH.

| Fluorescence units | Fluorescence units | |
|---|---|---|
| corresponding to bound | corresponding to bound | |
| biotinylated vesicles | biotinylated vesicles | |
| at pH = 7.4 | at pH = 6.5 | |
| 14,682 ± 110 | | |
| | 30,453 ± 222 | 1^{st} pH cycle from 7.4 to 6.5 |
| 14,712 ± 19 | | 2^{nd} pH cycle from 6.5 to 7.4 |
| | 29,064 ± 142 | 3^{rd} pH cycle from 7.4 to 6.5 |

***Fluorescence intensities of bound biotinylated fluorescent vesicles to streptavidin-covered magnetic microparticles are conserved after repeated pH cycles between the values of 7.4 and 6.5. The fluorescence intensities of non-biotinylated vesicles were 5,460 ± 121 and 5,698 ± 79 at pH 7.4 and 6.5, respectively. Errors correspond to standard deviations of repeated measurements (two samples per data point).

FIG. 5 shows that specific vesicle binding within the above PEG lipid content (closed symbols) increases with lowering pH, but no further increase in vesicle binding is observed for pH values lower than 6.7. This response does not imply that at pH values lower than 6.7 there is no further increase in the binding reactivity of biotinylated vesicles induced by further phase separation and further exposure of reactive groups. The biotin-streptavidin link that is utilized here as an example, is practically non-dissociative (Kₐ=10¹³ M⁻¹), and in principle, for the particular experimental setup, only one such contact between a vesicle and a streptavidin-coated microparticle would be adequate for vesicle immobilization on the microparticle. Therefore, it should be just that at pH values between 7.0 and 6.7 we observe the formation of transient free-of-PEG open spaces on the vesicle surface that reach the critical size required in order to accommodate docking of one streptavidin molecule. These studies are performed at equilibrium conditions by allowing twenty-four hours for the biotin-streptavidin bond to take place. Kinetically limited binding studies would affect the binding isotherms.

The observed pH-dependent response of lipid mixtures are attributable to the protonation of the carboxyl group of phosphatidylserine headgroups. The highest reported value for the apparent pKa of phosphatidylserine's carboxyl group is 5.5 at 0.1 ionic strength by determining the gel-to-fluid phase transition temperature of membranes composed of dimyristoyl- and dipalmitoyl-phosphatidylserine. The present invention illustrates measurable structural changes occurring at pH values higher by one logarithmic unit in membranes containing phosphatidylserine and cholesterol. The reported pKa value of 5.5 is underestimated in the sense that it is the mean value of the observed transition temperatures of the heating and cooling curves. In charged membranes, however, the direction of the thermal transition between an ordered phase and a fluid phase affects the effective surface charge density since the area per headgroup is different for the gel and the fluid phase. Ordered lipid phases due to smaller areas per headgroup have higher effective surface charge densities attracting higher concentrations of protons at the membrane surface governed by Boltzmann's law. This decreases the extent of dissociation of the carboxyl group at a given bulk pH, and, therefore, increases the value of the apparent pKₐ. In the present invention, the bilayer membranes do not exhibit thermal responses at temperatures at or lower than the working temperature of 37° C. suggesting a relatively condensed structure with higher surface charge density compared to the surface charge density in the fluid phase. The higher apparent pKₐ at which structural responses are observed in our studies could, therefore, be due to the underestimation of the reported pKₐ of the carboxyl group of phosphatidylserine in the gel phase and, potentially, due to even more ordered packing among lipids caused by the presence of cholesterol. This property of cholesterol is not unique to bilayers containing phosphatidylserine. Previous studies on bilayers containing phosphatidic acid show that cholesterol increases the apparent pH values at which membrane structural changes occur. In the absence of cholesterol, the same lipid mixture containing phosphatidylserine exhibits structural changes at lower pH values as indicated by thermal responses using DSC.

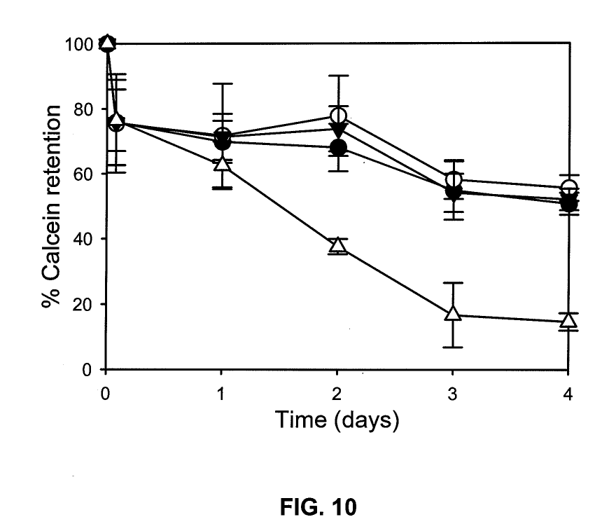

The observed M/E changes of pyrene-labeled lipid bilayers following the changes in pH are attributed to a great extent to changes in the membrane reorganization of the outer lipid leaflet for two reasons. First, the pH value of the encapsulated aqueous volume of vesicles was measured in parallel experiments by entrapping the fluorescent pH indicator HPTS and was found to be constant when transbilayer pH gradients were introduced (data not shown). In agreement with this suggestion is also the absence of membrane permeability to the encapsulated fluorescent compound calcein within the pH range of interest. However, calcein is larger than protons and its diffusion across the bilayer was measured along the opposite transbilayer direction for the same transmembrane pH gradients (see FIG. 10).

Referring to FIG. 10, for pH values above 5.5, encapsulated contents (calcein) are stably retained by vesicles composed of equimolar DPPC and DSPS lipids with 5% mol cholesterol and 0.5% mol DSPE-PEG lipids, incubated in phosphate buffer at 37° C. The initial drop in content retention during the first 10 minutes of incubation at all pH values studied is probably due to the fast change in temperature (from 25° C. to 37° C.) of the vesicle membranes that, possibly, respond by content release through melting of pH-independent defects. () pH=7.4; (∘) pH=6.7; (▾) pH=5.5; (∇) pH=4.0. Error bars correspond to standard deviations of repeated measurements (two vesicle preparations, three samples per preparation per time point). Changes in membrane permeability with decreasing pH were evaluated by monitoring the release from vesicles of encapsulated calcein at self-quenching concentrations (55 mM in pH=7.4).

In examples with pyrene-labeled lipid bilayers, it was observed that upon repeated cycles of lowering and increasing the pH of the outer lipid leaflet, these lipid bilayers exhibit a memory effect with a finite relaxation time. The possibility that this observation of memory in the membrane structure is due to reorganization of lipids in the inner lipid leaflet cannot be excluded. This response could be induced on the inner lipid leaflet by the pH-dependent heterogeneities formed on the outer lipid leaflet through acyl-tail interactions occurring at the hydrophobic interface between the lipid leaflets. Alternatively, repeated cycles of lowering and of increasing pH on the outer lipid leaflet of unilamellar vesicles may result in repeated assembly of lipids into heterogeneous domains followed by domain dispersion in laterally separated lipid aggregates of smaller but finite size, respectively. These smaller domains may act as nucleation points towards domain aggregation and fast formation of lipid heterogeneities during the next cycle of pH-induced protonation of DSPS lipids. Provision of adequate time for possible disassembly of these kinetically trapped domains may erase the observed memory on the membrane response. Such lipid reorganization changes may not be extensive enough to affect the emission shift of pyrene-labeled lipids.

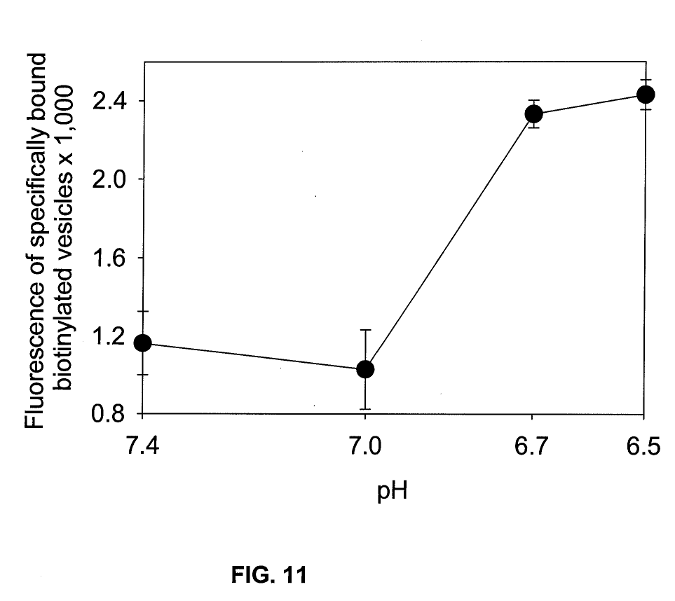

The property of the pH-dependent binding reactivity can be potentially utilized in vesicles as drug delivery carriers to solid tumors. These vesicles retain their pH-dependent binding response in the presence of 10% serum supplemented media by exhibiting approximately 100% increase in specific binding between pH 7.4 and 6.5 (see FIG. 11). Further engineering and optimization of these heterogeneous membranes could lead to useful technologies for the advancement of human health, particularly given the role of phosphatidylserine headgroups in promoting uptake by plasma membranes.

Referring to FIG. 11, vesicles composed of equimolar ratios of DPPC and DSPS lipids, 5% mol cholesterol and 0.5% mol DSPE-PEG lipids retain their pH-dependent binding reactivity towards streptavidin-coated magnetic microparticles in the presence of 10% serum supplemented media by exhibiting approximately 100% increase in specific binding between pH 7.4 and 6.5. Error bars correspond to standard deviations of repeated measurements (two vesicle preparations, at least two samples per time point).

### Illustrative Embodiment

### Targeting Cancer or Tumor Cell

Embodiments of this invention include pH-sensitive liposomes of a specific composition forming a stable structure that can efficiently carry biologically active agents. More particularly, the liposome can contain one or more biologically active agents, which can be administered into a mammalian host to effectively deliver its contents to and target a target cell or tumor cell. The liposomes can be capable of carrying biologically active agents such that the agents are sequestered in one environment and can be selectively exposed in another. Specifically, the use of a pH-tuned domain-forming membrane allows for tunable rigid-liposomes that can efficiently ‘expose’ the otherwise ‘hidden’ tumor-targeting ligands after liposome extravasation into tumors.

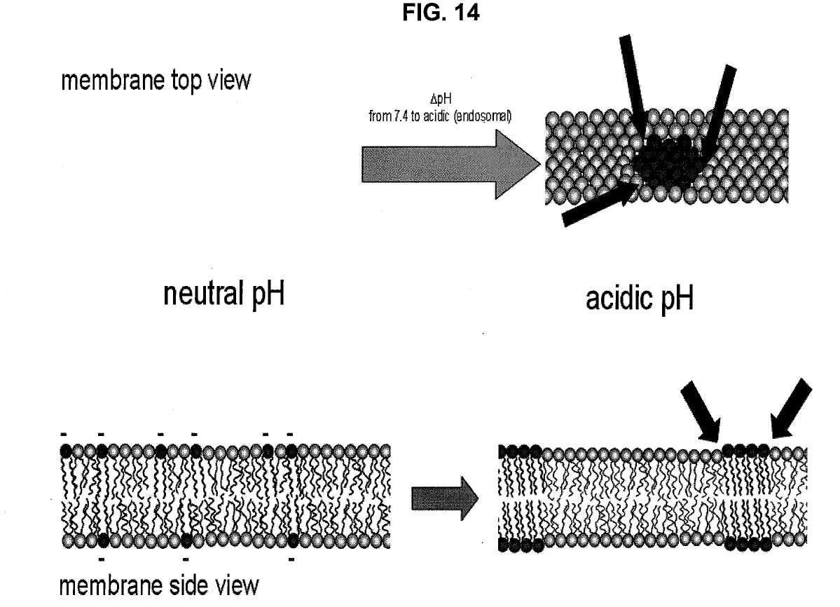

One aspect of this embodiment is to use pH-tuned liposomes as a mechanism to more efficiently and selectively expose the targeting ligand to the cancer cells composing a tumor or solid tumor. As shown in FIG. 14, which is a top and side view of pH-tunable liposomal membranes containing domain-forming lipids, the lipid-membrane surface appears homogeneous (mixed) at physiological pH (left) when electrostatic repulsion among the titratable anionic headgroups of domain-forming lipids is dominating (negative charges). At acidic pH (e.g. tumor interstitium 6.7) (right) protonation of the negatively charged headgroups allows the attractive Van der Waals forces among the hydrocarbon tails to dominate and lipid-separation and domain formation to occur. In general, under ‘raft’ or ‘domain’ hypothesis and as shown in FIG. 14, the long-saturated hydrocarbon-chains of phospholipids in membranes phase separate (aggregate in an ordered phase domain) in the plane of the membrane that should also contain lipids with hydrocarbon-chains of different or the same length.

Liposome Composition: One embodiment is a pH-sensitive liposome composition for targeting a biologically active agent to tumor cells, comprising:

a) at least two types of lipid phase separated domains formed by
i) a first lipid having a head group and a hydrophobic tail that, when protonated, is substantially miscible, wherein the first lipid is a zwitterionic lipid;
ii) a second lipid having a titratable charged head group, and a hydrophobic tail that, when protonated, is substantially immiscible with the first lipid;

b) a targeting ligand capable of binding an antigen or a marker and linked to the head group of a third lipid having a tail matching at least a portion of the first lipid or the second lipid,

wherein the liposome composition is adapted to laterally separate, via lipid phase separation, when the liposome composition is exposed to a specific environment, whereby the phase separation of the lipids exposes the targeting ligand to the more acidic environment.

Another embodiment is a liposome composition containing a biologically active agent, comprising:

a) at least two lipid phase separated domains formed by
i) a first lipid having a head group and a hydrophobic tail that, when protonated, is substantially miscible, wherein the first lipid is a zwitterionic lipid;
ii) a second lipid having a titratable charged head group, and a hydrophobic tail that, when protonated, is substantially immiscible with the first lipid;

b) a third lipid having a tail matching at least a portion of the first lipid or the second lipid, wherein the third lipid is PEG-linked; and

c) a targeting ligand capable of binding an antigen or a marker and linked to the headgroup of a fourth lipid having a tail matching at least a portion of the first lipid or the second lipid but not matching the tail of the PEG-linked third lipid,

wherein the liposome composition is adapted to laterally separate, via lipid phase separation, the PEG-linked third lipid from the targeting ligand-linked fourth lipid when the liposome composition is exposed to a specific environment, whereby the phase separation of the lipids exposes the targeting ligand to the more acidic environment. In one example, one of the first lipids is a zwitterionic lipid with one type of tail and one of the second lipids is a titratable head group lipid with a different type of tail. It is understood that additional lipids also can be incorporated into the composition.

In one embodiment, the liposomes can contain a targeting ligand attached to the surface of the PEG-coated liposomes. The targeting ligand can attach to the liposomes by direct attachment to liposome lipid surface components or through a short spacer arm or tether, depending on the nature of the moiety. A variety of methods are available for attaching molecules, for example, affinity moieties, to the surface of lipid vesicles. In one method, the targeting ligand is coupled to the lipid by a coupling reaction described below in the Examples, to form a targeting ligand-lipid conjugate, which conjugate is added to a solution of lipids for formation of liposomes. In another illustrative method, a vesicle-forming lipid activated for covalent attachment of a targeting ligand is incorporated into liposomes. The formed liposomes are exposed to the targeting ligand to achieve attachment of the targeting ligand to the activated lipids. One of ordinary skill in the art can select a method to attach a targeting ligand to the liposomes without undue experimentation.

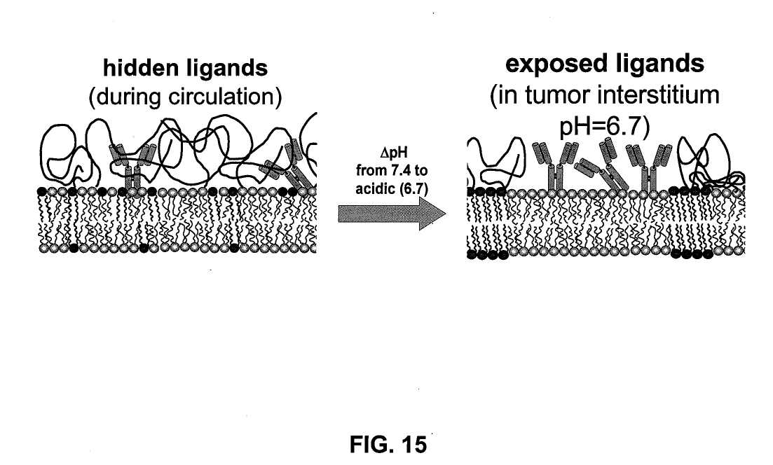

In another embodiment, the composition can selectively expose the targeting ligand to cancer cells (FIG. 15). For example, the targeting ligand is sterically obstructed by the neighboring PEG-linked lipids within the composition at a physiological (neutral) pH so that the composition can circulate in the blood steam (FIG. 15, left). As the liposome composition encounters the environment proximal to the tumor cell, which typically has a lower pH, the liposome lipid membrane forms lipid-separated domains, the neighboring PEG-linked lipids preferentially partition in lipid domains that are different from the lipid domains in which the ligand-linked lipids preferentially partition, and the targeting ligand is exposed to the tumor cell (FIG. 15, right). The exposed targeting ligand then may bind the tumor cell and deliver the biologically active agent.

Liposomes suitable for use in the composition include those composed primarily of vesicle-forming lipids. Such a vesicle-forming lipid is one that (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior, polar surface of the membrane. Many lipids suitable with this embodiment are of the type having two hydrocarbon chains, typically acyl chains, and a head group, either polar or charged. There are a variety of synthetic lipids and naturally forming lipids, including the phospholipids, such as DPPC, and DSPS (and DSPA), where the two hydrocarbon chains are typically at least 16 carbon atoms in length. The vesicle-forming lipids of this type are preferably ones having two hydrocarbon chains, typically acyl chains, and a head group, either polar or charged.

The pH-sensitive liposome can be selected to achieve a specified degree of fluidity or rigidity, to control the stability of the liposome in serum, to control the conditions effective for insertion of the targeting conjugate, to control the rate of ligand exposure for binding, and to control the rate of release of the entrapped biologically active agent in the liposome. Liposomes having a more rigid lipid bilayer, or a gel phase bilayer, are achieved by incorporation of a relatively rigid lipid, e.g., a lipid having a relatively high phase transition temperature, e.g., above about 39° C. Rigid, i.e., saturated, lipids contribute to greater membrane rigidity in the lipid bilayer. Other lipid components, such as cholesterol, are also known to contribute to membrane rigidity in lipid bilayer structures. In contrast, lipid fluidity can be achieved by incorporation of a relatively fluid lipid, typically one having a lipid phase with a relatively low liquid to gel crystalline phase transition temperature, e.g., at or below working temperature (e.g. body temperature).

These liposomes can contain titratable domain-forming lipids that phase-separate in the plane of the membrane as a response to decreasing pH values resulting in pH-controlled exposure of binding ligands for controlled targeting. In one embodiment, the liposomes are comprised of two lipid types (both T_{g}>37° C.): one type is a zwitterionic rigid lipid (e.g., dipalmitoyl phosphatidyl choline, DPPC, T_{g}=41° C.), and the other component is a ‘titratable domain-forming’ rigid lipid (e.g. distearoyl phosphatidylserin, DSPS, T_{g}=68° C.) that is triggered to phase-separate in the plane of the membrane as a response to decreasing pH values. At physiological pH (7.4) the lipid-headgroups of the ‘domain-forming’ rigid lipid (DSPS) are charged, electrostatic repulsion should prevail among DSPS lipids, and the liposomal membrane would appear more mixed and homogeneous, resulting in steric hindrance to binding of the ligand-linked lipids by the PEG-linked lipids, and in stable retention of encapsulated contents.

The lipid phase-separation can be tuned by introducing a titratable charge on the headgroups of the domain-forming lipids. The extent of ionization on the headgroups of the domain-forming lipids can be controlled by using the pH to adjust the balance between the electrostatic repulsion among the headgroups and the Van der Waals attraction among the hydrocarbon chains. The longer-hydrocarbon chain lipids that could phase-separate and form domains can be selected to have titratable acidic moieties on the head group (e.g., phosphatidyl serine). At neutral pH, the headgroups of these lipids are negatively charged opposing close approximation and formation of domains. As the pH is decreased, gradual head group protonation minimizes the electrostatic repulsion and lipid domains are formed.

In one embodiment, one of the lipids of the liposomes disclosed herein can have a negatively charged head group, and can have PEG-linked chains. The PEG-linked chains can help reduce the exposure of targeting ligands to other cells when in the blood stream. In this embodiment, the liposomes comprise ionizable ‘domain-forming’ (‘raft’-forming) rigid lipids that are triggered to form domains as a response to the tumor interstitial acidic pH. Domain formation (or else lateral lipid-separation) at the tumor interstitial pH can cause the targeting ligands to be ‘exposed’ due to lateral segregation of PEG-linked lipids in lipid domains that ligand-linked lipids do not preferentially partition. At physiological pH (during circulation) the lipids are charged, the liposome membrane may be ‘mixed’ so that the targeting ligands are ‘hidden’. At the acidic tumor interstitial pH (6.7-6.5), domain-forming lipids become increasingly protonated (non-ionized) and lipid domains of clustered protonated lipids can form resulting in exposure of targeting ligands. In one embodiment, the lipids can have a pK value between about 4 and about 7.

In another embodiment, one of the lipids of the liposomes disclosed herein can have a negatively charged head group, and can have PEG-linked chains. The PEG-linked chains can help reduce the likelihood of the liposome sticking to other cells when in the blood stream. In this embodiment, the liposomes comprise ionizable ‘domain-forming’ (‘raft’-forming) rigid lipids that are triggered to form domains as a response to the endosomal/lysosomal acidic pH. Domain formation (or else lateral lipid-separation) at the endosomal/lysosomal pH can cause the encapsulated contents to be released probably due to imperfections in ‘lipid packing’ around the domain ‘rim’. At physiological pH (e.g., during circulation) the contents cannot leak, as the lipids are charged and the liposome membrane may be ‘mixed’. At the acidic late endosomal/lysosomal pH (4.5-4.0), domain-forming lipids become increasingly protonated (non-ionized) and lipid domains of clustered protonated lipids can form resulting in release of encapsulated contents. In one embodiment, the lipids can have a pK value between about 3 and about 5.

In another embodiment, the liposomes disclosed herein may further comprise stabilizing agents or have an aqueous phase with a high pH. Examples of stabilizing agents are a phosphate buffer, an insoluble metal binding polymer, resin beads, metal-binding molecules, or halogen binding molecules incorporated into the aqueous phase to further facilitate retention of hydrophilic therapeutic modalities. Additionally, liposomes may comprise molecules to facilitate endocytosis by the target cells.

Liposomes can have a more rigid lipid bilayer, which can be achieved by the incorporation of a relatively rigid lipid. For example, lipids having a higher phase transition temperature tend to be more rigid. Further saturated lipids can contribute to greater membrane rigidity in the lipid bilayer. Other lipid components, such as cholesterol, are also known to contribute to membrane rigidity in fluid lipid bilayer structures.

In another embodiment, the liposomes can comprise rigid lipids (e.g. DPPC and DSPS), PEG-linked lipids and cholesterol or a cholesterol/sterol derivative. In one embodiment, liposomes were developed containing biotin-linked lipids with dipalmitoyl tails and PEG-linked lipids with distearoyl tails that contain the titratable DSPS domain-forming lipids that can be tuned to become activated at the slightly acidic conditions that corresponds to the tumor interstitial pH. Domain formation can potentially occur when both lipid constituents (both lamellar-forming) have long saturated rigid hydrocarbon-chains, but of different lengths. It has been found that using the pH-tuned domain-forming membranes is a mechanism to create tunable rigid-liposomes that will efficiently expose the otherwise ‘hidden’ tumor-targeting ligands after liposome extravasation in tumors.

In another embodiment, the ratio of DPPC to DSPS can range from about 9:1 to about 1:2, the cholesterol content can range from about 0-5% mole, the DSPE-PEG (2000 MW) can be equal or less than about 0.75-1.00% mole of total lipids and more than 0.25% mole of total lipids, and the biotinylated lipid can be equal or less than 1-2% mole of total lipids. In one example, rigid liposomes having DPPC (16:0), DSPS (18:0) and 5% mole cholesterol and 0.1-1.5% mole PEG (200 MW) were incubated in PBS at 37° C. at different pH values.

In another embodiment, the ratio of 21 PC to DSPS can range from about 9:1 about 1:2, the cholesterol content can range from about 0-5% mole, the DSPE-PEG (2000 MW) can be equal or less than about 5% mole of total lipids, and the biotinylated lipid can be equal or less than 1-2% mole of total lipids. In one example, rigid liposomes having 21PC (21:0), DSPS (18:0) and 5% mole cholesterol and 5% mole PEG (200 MW) were incubated in 10% serum supplemented media at 37° C. at different pH values.

Targeting Ligand. The liposomes optionally can be prepared to contain surface groups, such as antibodies or antibody fragments, small effector molecules for interacting with cell-surface receptors, antigens, and other like compounds, for achieving desired target-binding properties to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate. In one embodiment, the affinity molecule can be a complete antibody rather than a fragment of the antibody. While advances in antibody engineering can be employed to decrease immunogenic responses by the development of antibody fragments, tumor binding uptake and retention but for smaller fragments (Fab′, scFv) can decrease compared to the complete antibodies. These interactions can contribute to toxicities in vivo. These liposomes that are tuned to ‘hide’ antibodies during circulation and ‘expose’ the targeting ligands only in the close vicinity of cancer cells (within the acidic tumor-interstitium) can effectively address the issue of toxicity, and can reduce the issue of lower binding avidity of antibody fragments. By using the complete antibody, it is possible to achieve improved adhesion between the tumor cells and the liposomes.

Lipids can be derivatized with the targeting ligand by covalently attaching the ligand to the headgroup of a vesicle-forming lipid or to a short molecule (spacer arm or tether) already attached to the headgroup of a vesicle-forming lipid. There are a wide variety of techniques for attaching a selected ligand to a selected lipid headgroup. See, for example, Allen, T. M., et al., Biochemicia et Biophysica Acta 1237:99-108 (1995); Zalipsky, S., Bioconjugate Chem., 4(4):296-299 (1993); Zalipsky, S., et al., FEBS Lett. 353:71-74 (1994); Zalipsky, S., et al., Bioconjugate Chemistry, 705-708 (1995); Zalipsky, S., in Stealth Liposomes (D. Lasic and F. Martin, Eds.) Chapter 9, CRC Press, Boca Raton, Fla. (1995), which techniques are incorporated herein.

For example, the liposomes contain a targeting ligand, that effectively can bind specifically and with high affinity to a marker or target. In one example, the target can be the epithelial growth factor receptor family (EGFR), which is a common target for cancer therapy for solid tumors. Further, the targeting ligand can be a polypeptide or polysaccharide effector molecule capable of binding a marker on solid tumor cell. Affinity moieties, suitable with this invention, can be found in current and future literature.

Other targeting ligands are well known to those of skill in the art, and in other embodiments, the ligand is one that has binding affinity to epithelial tumor cells, and which is, more preferably, internalized by the cells. Such ligands often bind to an extracellular domain of a growth factor receptor. Exemplary receptors (epitopes) on cancer cell surfaces include the epidermal growth factor receptor (EGFR), the folate receptor, the transferrin receptor (CD71), ErbB2, and the carcinoembryonic antigen (CEA).

Biologically Active Agents. In one embodiment, the liposomal encapsulation of a biologically active agent enhances the bioavailability of the modalities in cancer cells. In this embodiment, the liposome can be used to encapsulate a biologically active agent (e.g., cancer therapeutic modalities) and efficiently release the therapeutic modality in cancer cells, thus allowing toxicity to occur in the tumor cells. For example, the use of pH sensitive liposome allows more complete release of the therapeutic modalities upon endocytosis by the cancer cell and into the late endosomal or lysosomal compartment.

The liposome can have a phospholipid-membrane rigidity to improve the retention of the bioactive agent in the liposome during blood circulation. The addition of PEG-linked lipids also reduces liposome clearance, thus increasing liposome accumulation in tumors. For example, one embodiment includes a pH-sensitive liposome with rigid membranes that combine long circulation times with the release of contents in the late endosome or lysosome. Other types of pH-sensitive liposomes can include charged titratable peptides on the surface that can cause phase separation and domain formation on charged membranes.

This invention further relates to a novel liposome structure capable of carrying bioactive agents. For example, this invention provides an improved liposome formulation and a nucleic acid, which can produce high levels of gene expression and protein production. Further, targeted α-particle emitters hold great promise as therapeutic agents for targeted cancer therapy, and can be delivered by liposomes. Other bioactive agents suitable with this invention are obvious to those with ordinary skill in the art and can be researched without undue experimentation.

Other biologically active agents suitable with such liposomes include but are not limited to natural and synthetic compounds having the following therapeutic activities: anti-arthritic, anti-arrhythmic, anti-bacterial, anticholinergic, anticoagulant, antidiuretic, antidote, antiepileptic, antifungal, anti-inflammatory, antimetabolic, antimigraine, antineoplastic, antiparasitic, antipyretic, antiseizure, antisera, antispasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic, and vasodilating. In one illustrative example, the entrapped agent is a cytotoxic drug, that is, a drug having a deleterious or toxic effect on cells.

Administration of Liposome Composition. Liposomes can be used as drug delivery carriers for therapy of metastatic cancer, and other inflammatory types of diseases, and also as delivery vehicles for vaccines, gene therapy, etcetera. The present invention further provides an effective vaccine vehicle capable of effective delivery, boosting antigen-immune response and lowering unwanted extraneous immune response, presently experienced with adjuvants. The liposomes according to the invention may be formulated for administration in any convenient way. The invention therefore includes within its scope pharmaceutical compositions comprising at least one liposomal compound formulated for use in human or veterinary medicine. Other routes of administration will be known to those of ordinary skill in the art and can be readily used to administer the liposomes of the present invention.

Another embodiment of this invention includes a method comprising pre-injecting the individual with empty liposomes and saturating the reticuloendothelial organs to reduce non-tumor specific spleen and liver uptake of the liposome-encapsulated therapeutics upon administration thereof.

In use and application, the liposome can be used to preferentially deliver a biologically active agent to a target cell or cancer cell of vascularized (solid) tumors. For example, in drug delivery to metastatic tumors with developed vasculature, the preferential tumor accumulation and retention of liposomes is primarily dependent on their size (EPR effect), and can result in adequate tumor adsorbed doses that can be further enhanced by ‘switching on’ the specific targeting of cancer cells after liposome extravasation into the tumor interstitium.

The liposome of the invention may be formulated for parenteral administration by bolus injection or continuous infusion. Formulation for injection may be presented in unit dosage form in ampoules, or in multi-dose containers with an added preservative. The compositions may take such forms as suspensions, solutions or emulsions in oily or aqueous vehicles, and may contain formulatory agents such as suspending, stabilizing, and/or dispersing agents. Alternatively, the active ingredient may be in powder form for reconstitution with a suitable vehicle, e.g., sterile pyrogen-free water, before use.

One embodiment of the invention includes a method for administering a biologically active agent comprising selecting a liposome comprising at a least first rigid lipid and a second rigid lipid each having a head group and a hydrophobic tail, wherein the lipids when both protonated are not particularly miscible, and a polyethyleneglycol-linked lipid having a side chain matching at least a portion of the first or the second lipid, wherein the first lipid is a zwitterionic lipid and the second lipid has a titratable head group; and the composition is adapted to ‘expose’ targeting ligands at a certain pKa, and to release an entrapped biologically active agent at a certain pKa of lower value; preparing a liposome composition with the at least the first rigid lipid and the second rigid lipid and the polyethyleneglycol-linked lipid; preparing a therapeutic liposome by combining the composition with the biologically active agent so that the biologically active agent is within the liposome composition whereby the therapeutic liposome is adapted to release the entrapped biologically active agent at a certain pKa of lower value; and administering the therapeutic liposome to a subject.

The liposomes according to the invention may be formulated for administration in any convenient way. The invention therefore includes within its scope pharmaceutical compositions comprising at least one liposomal compound formulated for use in human or veterinary medicine. Such compositions may be presented for use with physiologically acceptable carriers or excipients, optionally with supplementary medicinal agents. Conventional carriers can also be used with the present invention.

Overcoming Immune Response. To overcome immunogenicity, in one embodiment of the invention the liposomes are modified with PEG-linked lipids for use with the specific organism. In another embodiment, a method further comprises coating the outer membrane surfaces of the liposomes with molecules that preferentially associate with a specific target cell. These molecules or targeting agents may be antibodies, peptides, engineered molecules, or fragments thereof.

For example, to achieve tumor targeting of ovarian and breast cancer cells and internalization, liposomes can be coated (immunolabeled) with Herceptin, a commercially available antibody that targets antigens that are over-expressed on the surface of such cancer cells. Herceptin is chosen to demonstrate proof of principle with the anticipation that other antibodies, targeting ovarian, breast, liver, colon, prostate and other carcinoma cells could also be used. The target cells may be cancer cells or any other undesirable cell. Examples of such cancer cells are those found in ovarian cancer, breast cancer or metastatic cells thereof. The active targeting of liposomes to specific organs or tissues can be achieved by incorporation of lipids with monoclonal antibodies or antibody fragments that are specific for tumor associated antigens, lectins, or peptides attached thereto.

Because the biologically active agent is sequestered in the liposomes, targeted delivery is achieved by the addition of peptides and other ligands without compromising the ability of these liposomes to bind and deliver large amounts of the agent. The ligands are added to the liposomes in a simple and novel method. First, the lipids are mixed with the biologically active agent of interest. Then ligands either chemically become conjugated on the head groups of some of the lipids or ligand-linked lipids are added directly to the liposomes.

For other biologically active agents that need to be actively loaded into preformed liposomes, decoration of liposomes with targeting ligands can occur either before loading of preformed liposomes with the biologically active agents or after.

Preparing Liposomes. The liposomes may be prepared by a variety of techniques, such as those detailed in Lasic, D. D., Liposomes from Physics to Applications, Elsevier, Amsterdam (1993), which techniques are incorporated herein. Specific examples of liposomes prepared in support of the present invention will be described herein. Typically, the liposomes can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates have sizes between about 0.1 to 10 microns.

After formation, the liposomes are sized. One more effective sizing method for liposomes involves extruding an aqueous suspension of the liposomes through a series of polycarbonate membranes having a selected uniform pore size in the range of 0.03 to 0.2 micron, typically about 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less. In one embodiment of the present invention, the liposomes are extruded through polycarbonate filters with pore size of 0.1 μm resulting in liposomes having diameters in the approximate range of about 120 nm.

Incorporating Biologically Active Agent into Liposomes. The biologically active agent of choice can be incorporated into liposomes by standard methods, including passive entrapment of a water-soluble compound by hydrating a lipid film with an aqueous solution of the agent, passive entrapment of a lipophilic compound by hydrating a lipid film containing the agent, and loading an ionizable drug against an inside/outside liposome pH gradient. Other methods, such as reverse evaporation phase liposome preparation, are also suitable.

Another embodiment includes a method of formulating a therapeutic liposome composition having sensitivity to a target cell. The method includes selecting a liposome formulation composed of pre-formed liposomes comprising at least a first lipid and a second lipid each having a head group and a hydrophobic tail, wherein the lipids when both protonated are not particularly miscible, and containing PEG-linked lipids of one type of tails, and having an entrapped biologically active agent; selecting from a plurality of targeting conjugates a targeting conjugate composed of a lipid having a polar head group and a hydrophobic tail of the other type than that of the PEG-linked lipid's, and a targeting ligand attached to the headgroup of the lipid; and combining the liposome formulation and the selected targeting conjugate to form a therapeutic, target-cell pH sensitive liposome composition.

Kits. The present invention includes kits containing the present liposome structure capable of carrying a reagent within it. One such kit may comprise the liposome structures ready for the user to add the biological reagent of interest. A kit may further comprise a liposome preparation and one or more specific biologically-active reagents for addition to the liposome structure. Another kit of the present invention comprises a set of liposome structures, each containing a specific, biologically-active reagent, which when administered together or sequentially, are particularly suited for the treatment of a particular disease or condition.

### EXAMPLES

### Example 1

Biotinylated liposomes (1% mole DPPE-biotin) were developed containing PEGylated lipids that contain domain-forming lipids, which were tuned to become activated at conditions similar to those of tumor interstitial pH. Rigid liposomes consisting of DPPC (16:0), DSPS (18:0) (at 1:1 mole ratios), and 5% cholesterol and 0.1% to 1.5% mole DSPE-PEG (2000 MW) were incubated in PSB at 37° C. at various pH values.

### Example 2

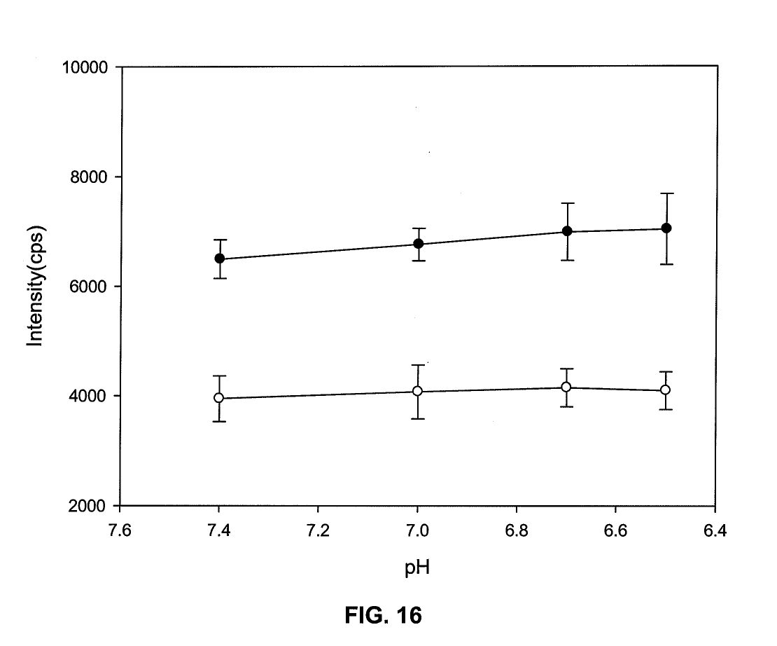

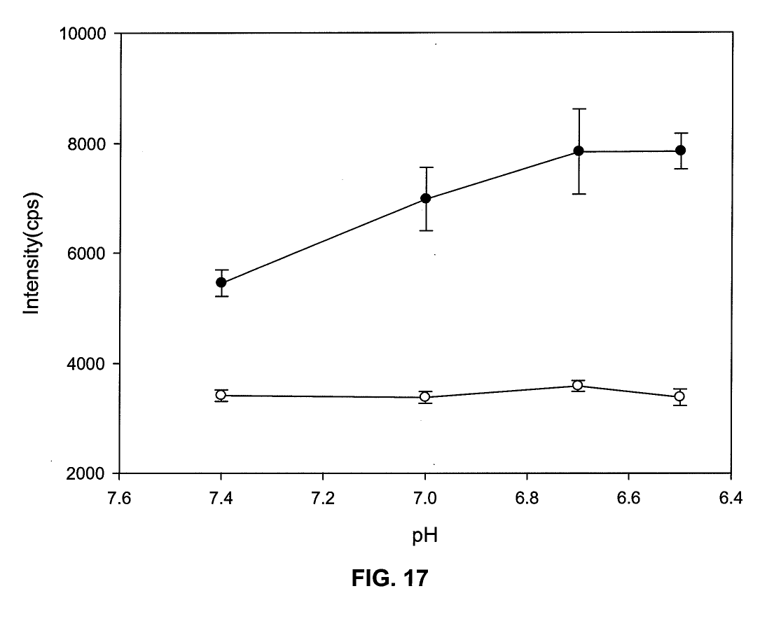

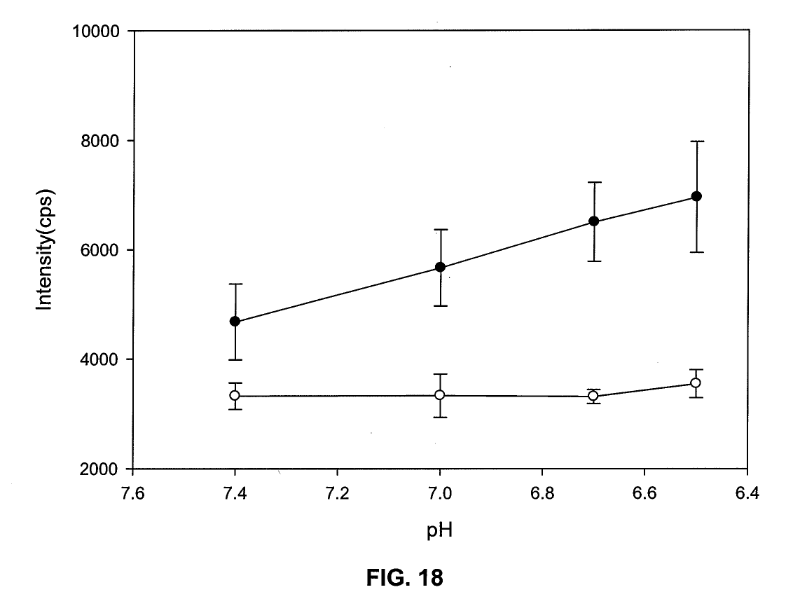

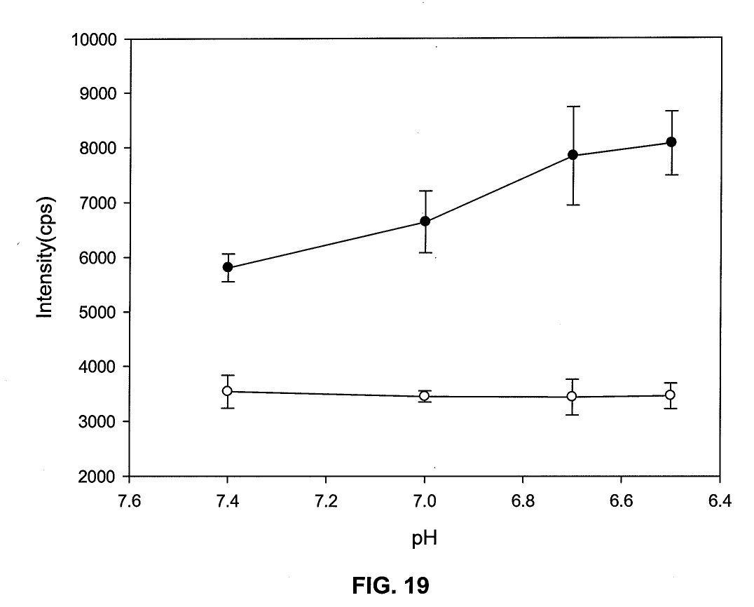

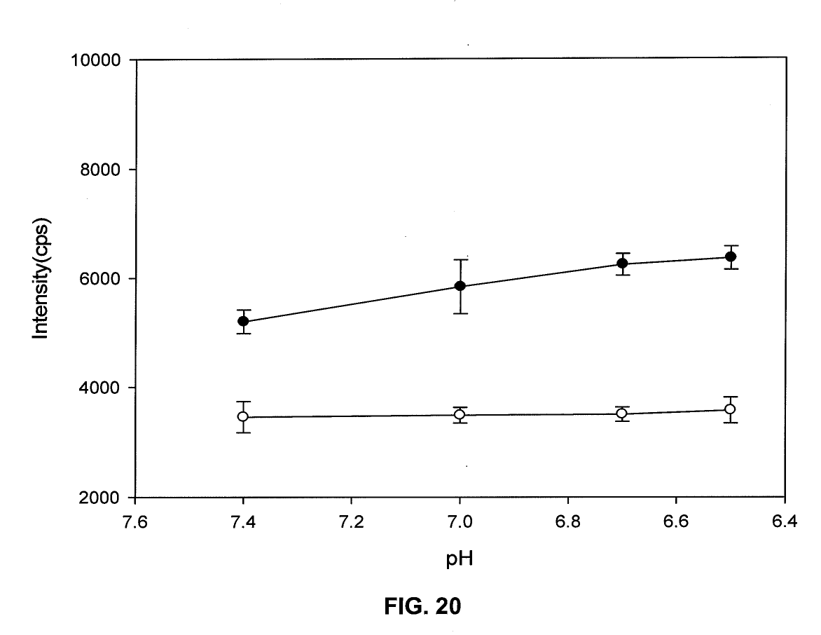

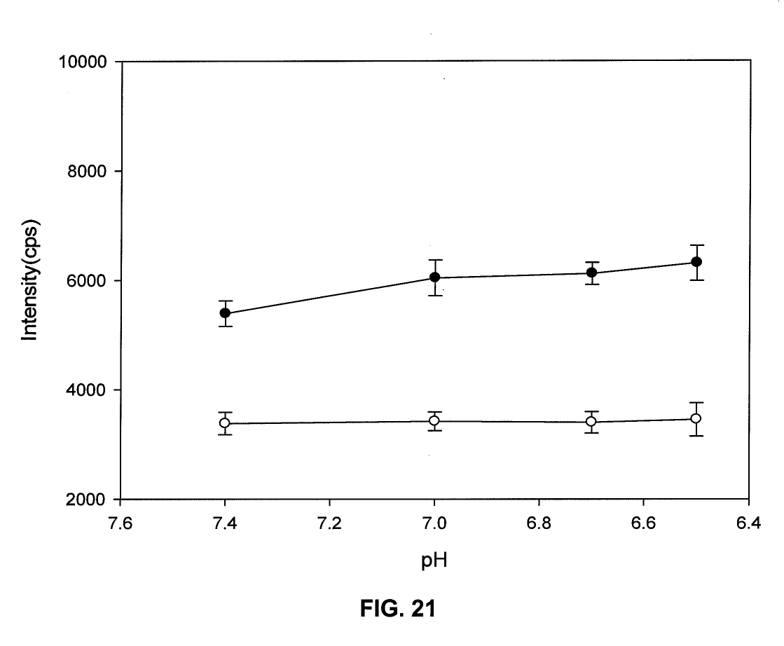

Binding of rigid biotinylated liposomes (FIGS. 16, 17, 18, 19, 20, 21, filled symbols) to streptavidin-covered-magnetic microbeads was evaluated at various pH values ranging from pH 7.4, approximating the pH of the blood during circulation of liposomes to pH 6.5 that corresponds to the pH of the tumor interstitium after extravasation of liposomes into the tumor. The extent of liposomes bound was evaluated for different amounts of PEG-linked lipids in the liposome composition ranging from 0.1% to 1.5% mole (of total lipid) and was also compared to identical liposomes without biotin (plain liposomes) indicated by the open symbols in FIGS. 16, 17, 18, 19, 20, and 21. In biotinylated liposomes the amount of biotin-linked lipids was retained constant at 1% mole of total lipid (FIG. 16 shows liposomes containing 0.1% mole PEG-linked lipid, FIG. 17 0.25% mole, FIG. 18 0.5% mole, FIG. 19 0.75% mole, FIG. 20 1.0% mole, and FIG. 21 1.5% mole). The liposomal membrane was labeled with rhodamine, and liposomes were allowed to bind to the magnetic beads and after ten successive magnetic separations and washings with PBS, the magnetic beads were incubated in fresh PBS (pH=7.4) with Triton-X 100 to release the bound lipids that were then quantitated by measuring their fluorescence intensity. An increase in fluorescence intensity (cps), with a decrease in the pH of the incubation environment during binding, showed that the affinity marker or target ligand was exposed in the lower pH environment.

For fractions of PEG-linked lipids ranging between 0.25% and 0.75% mole, the specific binding efficacy shows a sharp transition within the narrow pH values of the physiological pH=7.4 and the tumor interstitial pH=6.7 (FIGS. 17, 18, 19). Depending on the molecular size (length) of the targeting ligand (defined as the distance that the binding moiety extends from the physical surface of the liposome), using trial and error, the fraction of PEG-linked lipids that have to be included in the lipid composition to maximize the increase in specific binding between pH 7.4 and 6.5. To optimize the conditions for maximum binding between different pH values, the pKa of the ionized titratable lipid was adjusted.

### Example 3

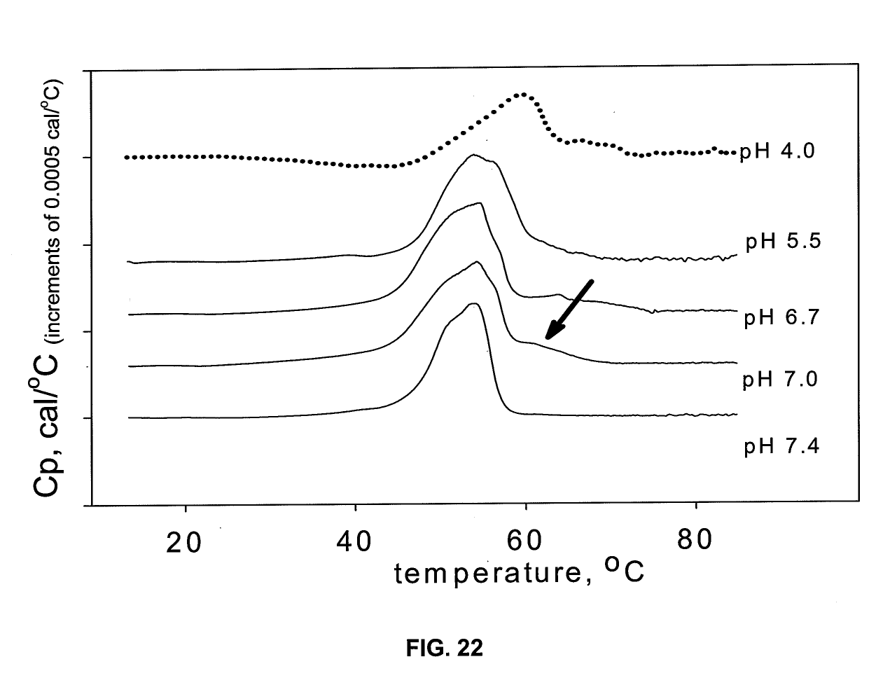

Differential Scanning Calorimetry (DSC) was used because it can provide direct evidence of phase separation of lipid membranes. FIG. 22 shows the thermal scans of the same liposome composition (equimolar DPPC and DSPS with 5% mole cholesterol and 2% mole DSPE-PEG), performed at a rate of 60° C./h. As the pH was decreased from 7.4 to 4.0, an enhancement was observed on the contributions from thermal transitions at higher temperatures. Higher thermal transitions at lower pH values suggest increasing formation of lipid phases that are rich in clustered (protonated) DSPS lipids (that has higher Tg) and phases poor in DSPS lipids (or richer in DPPC lipids, FIG. 22). These results demonstrate that in membranes containing lipids with different hydrocarbon chain lengths (with one lipid type bearing charged headgroups), lipid mixing or domain formation is controlled by the pH that affects the extent of electrostatic repulsion among the titratable lipids.

### Example 4

The release of encapsulated fluorescent contents, specifically in this example calcein, from PEGylated liposomes, composed of equimolar ratios of DPPC and DSPS was investigated by calcein quenching efficiency measurements. The lipid film was hydrated in 1 ml phosphate buffer containing 55 mM calcein (pH 7.4, isosmolar to PBS). The unentrapped calcein was removed at room temperature by size exclusion chromatography (SEC) using a Sephadex G-50 column (of 11 cm length) and was eluted with phosphate buffer (1 mM EDTA, pH=7.4). To evaluate the release of calcein from the liposomes, the liposomes containing self-quenching concentrations of calcein (55 mM) were incubated in phosphate buffer at different pH values at 37° C. over time. The concentration of lipids for incubation was 0.20 μmoles/ml.

The release of calcein from the liposomes and its dilution in the surrounding solution resulted in an increase in fluorescence due to relief of self-quenching. Calcein release was measured at different time points by adding fixed quantities of liposome suspensions into cuvettes (1 cm path length) containing phosphate buffer (1 mM EDTA, pH 7.4). Calcein fluorescence (ex: 495 nm, em: 515 nm) before and after addition of Triton-X 100, was measured using a Fluoromax-2 spectrofluorometer (Horiba Jobin Yvon, N.J.), and was used to calculate the quenching efficiency defined as the ratio of fluorescence intensities after and before addition of Triton-X 100. The percentage of retained contents with time was calculated as follows:

$$\% calcein retention = ( \frac{{Q}_{t} - {Q}_{min}}{{Q}_{max} - {Q}_{min}} ) \times 100$$

where, Qₜ is calcein quenching efficiency at the corresponding time point t, Qₘₐₓ is the maximum calcein quenching efficiency in phosphate buffer (at pH 7.4) at room temperature immediately after separation of liposomes by SEC, and Qₘᵢₙ is the minimum quenching efficiency equal to unity.

FIG. 10 shows the percentage of calcein retention as a function of pH {pH 7.4 (), pH 5.5 (∘), pH 5.0 (▾), pH 4.0 (∇)} by liposomes composed of equimolar DPPC and DSPS (with 5% mole cholesterol and 2% mole PEGylated lipids), incubated in PBS at 37° C. FIG. 18 shows the content release over 5 days. The error bars correspond to standard deviations of repeated measurements of two liposome preparations, two samples per preparation per time point. The initial drop in content retention during the first 10 minutes of incubation is probably due to osmotic and temperature differences between the encapsulated and surrounding solutions. After the first 10 minutes, encapsulated contents are stably retained by liposomes, and effectively released at the acidic pH=4 that corresponds to late endosomal lysosomal values, indicating that these liposomes can effectively release their therapeutic cargo after specific binding and endosomal internalization by target cells.

The foregoing detailed description of the preferred embodiments and the appended figures have been presented only for illustrative and descriptive purposes. They are not intended to be exhaustive and are not intended to limit the scope and spirit of the invention. The embodiments were selected and described to best explain the principles of the invention and its practical applications. One skilled in the art will recognize that many variations can be made to the invention disclosed in this specification without departing from the scope and spirit of the invention.

What is claimed is:

1. A method for controlling the surface topography and binding reactivity in functionalized lipid layers, including in the form of liposomes, comprising the steps of:
a) providing a first lipid having a headgroup and a tail, wherein at least a first portion of the first lipid is domain forming with titratable anionic headgroups and at least a second portion of the first lipid comprises grafted hydrophilic polymer chains attached to the headgroup;
b) providing a non-ionizable second lipid comprising hydrocarbon tails different from or the same as the tail of the first lipid;
c) providing a third lipid comprising grafted functional groups attached to the headgroup and hydrocarbon tails identical to the tail of the second lipid;
d) producing a lipid bilayer membrane from the first, second and third lipids;
e) subjecting the lipid bilayer structure to an environment having a first pH, wherein at the first pH, the lipids form a generally homogenous functionalized lipid bilayer; and
f) lowering the pH of the environment to a second pH, wherein at the second pH, the functionalized lipid bilayer is reorganized into lipid heterogeneities;
wherein the surface topography and binding reactivity in the functionalized lipid bilayers at the second pH are different from the surface topography and binding reactivity in the functionalized lipid layers at the first pH.

2. The method as claimed in claim 1, wherein lowering the pH creates lipid phase separation on the membrane.

3. The method as claimed in claim 2, wherein at the first pH, the first lipid headgroups are charged and repulsion between the headgroups makes the lipid energetically less likely to crystallize.

4. The method as claimed in claim 3, wherein at the first pH, the lipid bilayer is spatially less heterogeneous, and the functional groups are obstructed by surrounding polymer chains.

5. The method as claimed in claim 4, wherein at the second pH, the headgroups of the first lipid become protonated, reducing electrostatic repulsion and increasing hydrogen bonding between the protonated first lipid headgroups and a portion of at least a second portion of the first lipid comprising grafted hydrophilic polymer chains attached to the headgroup, whereby at the lower pH the first lipids partition into protonated lipid heterogeneities.

6. The method as claimed in claim 5, whereby the second and third lipids partition into different lipid heterogeneities, driven by the dispersive attractive forces between the hydrocarbon tails of the second and third lipids.

7. The method as claimed in claim 6, whereby at the lower pH the third lipids become exposed and available to interact with targets thereby increasing the effective binding reactivity of membranes.

8. The method as claimed in claim 7, wherein the surface topography of the lipid bilayer is remodeled upon the lowering of the pH.

9. The method as claimed in claim 8, wherein the formation of the heterogeneities is reversible.

10. The method as claimed in claim 1, wherein the domain forming first lipid is DSPS, the first lipid with grafted polymer chains is DSPE-PEG, the second lipid is DPPC, and the third lipid is DPPE-biotin.

11. The method as claimed in claim 1, further comprising at least two lipid phase separated domains formed by:
i) the first lipid, which when protonated is substantially miscible; and
ii) the second lipid, which further comprises a titratable charged head group and a hydrophobic tail and when protonated is substantially immiscible with the first lipid.

12. The method as claimed in claim 11, wherein the third lipid is PEG-linked.

13. The method as claimed in claim 1, further comprising a targeting ligand capable of binding an antigen or a marker and linked to the headgroup of a fourth lipid having a tail matching at least a portion of the first lipid or the second lipid but not matching the tail of the third lipid, wherein the lipid composition is adapted to laterally separate, via lipid phase separation, the third lipid from the targeting ligand-linked fourth lipid when the liposome composition is exposed to a specific environment, whereby the phase separation of the lipids exposes the targeting ligand to the specific environment.

14. The method as claimed in claim 13, wherein the specific environment is an acidic environment.

15. The method as claimed in claim 1, wherein the lipids have phase transition temperatures above 37° C.

16. The method as claimed in claim 1, wherein the lipids bear a negative charge at a neutral pH.

17. The method as claimed in claim 1, wherein 0 to 10% cholesterol is included in the lipid composition.

18. The lipid composition as claimed in claim 1, wherein when the lipids are protonated, they are not substantially miscible and form more than one lipid phase-separated domain.

19. A lipid composition occurring in complex bilayer membranes in the form of vesicles comprising:
a) a first lipid having a headgroup and a tail, wherein at least a first portion of the first lipid is domain forming with titratable anionic headgroups and at least a second portion of the first lipid comprises grafted polymer chains attached to the headgroup;
b) a non-ionizable second lipid comprising hydrocarbon tails that are different from or the same as the tail of the first lipid; and
c) a third lipid comprising grafted functional groups attached to the headgroup and hydrocarbon tails identical to the tail of the second lipid,
wherein a lipid bilayer membrane is produced from the first, second and third lipids, the lipid bilayer structure being subjected to an environment having a first pH, wherein at the first pH, the lipids form a generally homogenous functionalized lipid bilayer, and the pH of the environment being lowered to a second pH, wherein at the second pH, the functionalized lipid bilayer is reorganized into lipid heterogeneities;
whereby the surface topography and binding reactivity in the functionalized lipid bilayers at the second pH are different from the surface topography and binding reactivity in the functionalized lipid layers at the first pH.

20. The lipid composition as claimed in claim 19, wherein the first lipid headgroups are charged.

21. The lipid composition as claimed in claim 19, wherein the domain forming first lipid is DSPS, the first lipid with grafted polymer chains is DSPE-PEG, the second lipid is DPPC, and the third lipid is DPPE-biotin.

22. The lipid composition as claimed in claim 19, further comprising at least two lipid phase separated domains formed by:
i) the first lipid, which when protonated is substantially miscible; and
ii) the second lipid, which further comprises a titratable charged head group and a hydrophobic tail and when protonated is substantially immiscible with the first lipid.

23. The lipid composition as claimed in claim 22, wherein the third lipid is PEG-linked.

24. The lipid composition as claimed in claim 19, further comprising a targeting ligand capable of binding an antigen or a marker and linked to the headgroup of a fourth lipid having a tail matching at least a portion of the first lipid or the second lipid but not matching the tail of the third lipid, wherein the lipid composition is adapted to laterally separate, via lipid phase separation, the third lipid from the targeting ligand-linked fourth lipid when the liposome composition is exposed to a specific environment, whereby the phase separation of the lipids exposes the targeting ligand to the specific environment.

25. The lipid composition as claimed in claim 24, wherein the specific environment is an acidic environment.

26. The lipid composition as claimed in claim 19, wherein the lipids have phase transition temperatures above 37° C.

27. The lipid composition as claimed in claim 19, wherein the lipids bear a negative charge at a neutral pH.

28. The method as claimed in claim 19, wherein 0 to 10% cholesterol is included in the lipid composition.

29. The lipid composition as claimed in claim 19, wherein when the lipids are protonated, they are not substantially miscible and form more than one lipid phase-separated domain.
